(19) United States
(12) Patent Application Publication
(10) Pub. No.: US 2025/0076282 A1
(43) Pub. Date: Mar. 6, 2025

(54) Identification of Viable Human Embryos

(71) Applicant: Overture Life, Inc., New York, NY (US)

(72) Inventors: José Antonio Horcajadas Almansa, Madrid (ES); Hussain A. Abdulla, Corpus Christi, TX (US)

(73) Assignee: Overture Life, Inc., New York, NY (US)

(21) Appl. No.: 18/951,011

(22) Filed: Nov. 18, 2024

Related U.S. Application Data
(63) Continuation of application No. 16/937,692, filed on Jul. 24, 2020, now Pat. No. 12,181,462.
(60) Provisional application No. 62/878,524, filed on Jul. 25, 2019.

(51) Int. Cl. G01N 33/50

(52) U.S. Cl. CPC G01N 33/5005

(57) ABSTRACT

Disclosed herein is a method for assessing the probability of embryo implantation success during the in vitro fertilization process. A method of the disclosure can predict embryo implantation success based on, for example, the amount of specific metabolites present in embryo conditioned culture media.

### CROSS-REFERENCE

This application is a continuation of U.S. patent application Ser. No. 16/937,692 filed Jul. 24, 2020, which claims the benefit of U.S. Provisional Application No. 62/878,524, filed Jul. 25, 2019, each of which is incorporated herein by reference in its entirety.

### BACKGROUND

Assisted reproductive technologies (ARTs) such as in vitro fertilization (IVF) provide infertile couples with an opportunity to achieve a pregnancy. Today, over 1% of all infants born in the United States and up to 5-8% born in some European countries are conceived using ART.

### INCORPORATION BY REFERENCE

Each patent, publication, and non-patent literature cited in the application is hereby incorporated by reference in its entirety as if each was incorporated by reference individually.

### SUMMARY OF THE INVENTION

In some embodiments, the disclosure provides a method comprising: a) measuring an amount of a metabolite in a sample of culture media in which an embryo has been cultured in vitro, wherein the metabolite is caprolactam, N-cyclohexylformamide, threo-3-hydroxy-2-methylbutyric acid, sulfoacetic acid, (2,7-dimethyloctahydro-1H-cyclopenta[c]pyridin-4-yl)methanol, trans-3-indoleacrylic acid, pantothenic acid, diaveridine, (5-benzyl-3,6-dioxo-2-piperazinyl)acetic acid, dasytrichone, indole, leucoline, skatole, 6-hydroxycaproic acid, 1,2,6-hexanetriol, naphthalen-2-amine, 8-hydroxyquinoline, 2,2,6,6-tetramethyl-4-piperidinol, 1,5-naphthalenediamine, 1H-indole-2-carboxylic acid, (E)-dacarbazine, dinitrosopentamethylenetetramine, 5-hydroxyindole-3-acetic acid, 3-amino-5,7-dimethyl-1-adamantanol, 11-aminoundecanoic acid, kynurenine, N-hydroxy-L-tryptophan, 2-(methylamino)-1H-benzo[de]isoquinoline-1,3(2H)-dione, 5-hydroxy-N-formylkynurenine, or N-lauroylglycine; and b) assessing a likelihood of success of implantation of the embryo into a uterus upon introduction of the embryo into the uterus based at least partially on the amount of the metabolite present in the sample of culture media.

In some embodiments, the disclosure provides a method comprising measuring an amount of each of a plurality of metabolites in a mixture, wherein the metabolites is (2,7-dimethyloctahydro-1H-cyclopenta[c]pyridin-4-yl)methanol, trans-3-indoleacrylic acid, pantothenic acid, diaveridine, (5-benzyl-3,6-dioxo-2-piperazinyl)acetic acid, dasytrichone, indole, skatole, 8-hydroxyquinoline, (E)-dacarbazine, kynurenine, or N-hydroxy-L-tryptophan.

In some embodiments, the disclosure provides a method comprising: a) performing an assay to obtain a first set of mass spectra corresponding to a first metabolite present in a sample of culture media in which an embryo has been cultured in vitro (ms₁); b) using the first set of mass spectra to determine an amount of the first metabolite in the sample of culture media, wherein the amount of the first metabolite is associated with a low probability of embryo implantation success; c) performing an assay to obtain a second set of mass spectra corresponding to a second metabolite present in the sample of culture media in which the embryo has been cultured in vitro (ms₂); d) using the second set of mass spectra to determine an amount of the second metabolite, wherein the amount of the second metabolite is associated with a high probability of embryo implantation success; and e) comparing the amount of the first metabolite with the amount of the second metabolite.

In some embodiments, the disclosure provides a method comprising: a) measuring an amount of each of four metabolites in a sample of culture media in which an embryo has been cultured in vitro; and b) assessing a likelihood of success of implantation of the embryo into a uterus upon introduction of the embryo into the uterus based on the amount of the 4 metabolites in the sample of culture media, wherein: i) the amount of at least one of the metabolites in the sample of culture media is associated with a low probability of embryo implantation success; and ii) the amount of at least one of the metabolites in the sample of culture media is associated with a high probability of implantation success.

In some embodiments, the disclosure provides a method comprising a) requesting an assay on a sample of culture media in which an embryo has been cultured in vitro, b) receiving a communication, wherein the communication assesses a likelihood of success of implantation of the embryo into a uterus upon introduction of the embryo into the uterus based on an amount of one or more metabolites measured in the sample of culture media; and c) transferring the embryo into a subject's uterus, wherein: i) the communication states that the likelihood of success of implantation of the embryo into the uterus is high; and ii) one of the metabolites measured in the sample of culture media is caprolactam, N-cyclohexylformamide, threo-3-hydroxy-2-methylbutyric acid, sulfoacetic acid, (2,7-dimethyloctahydro-1H-cyclopenta[c]pyridin-4-yl)methanol, trans-3-indoleacrylic acid, pantothenic acid, diaveridine, (5-benzyl-3,6-dioxo-2-piperazinyl)acetic acid, dasytrichone, indole, leucoline, skatole, 6-hydroxycaproic acid, 1,2,6-hexanetriol, naphthalen-2-amine, 8-hydroxyquinoline, 2,2,6,6-tetramethyl-4-piperidinol, 1,5-naphthalenediamine, 1H-indole-2-carboxylic acid, (E)-dacarbazine, dinitrosopentamethylenetetramine, 5-hydroxyindole-3-acetic acid, 3-amino-5,7-dimethyl-1-adamantanol, 11-aminoundecanoic acid, kynurenine, N-hydroxy-L-tryptophan, 2-(methylamino)-1H-benzo[de]isoquinoline-1,3(2H)-dione, 5-hydroxy-N-formylkynurenine, or N-lauroylglycine.

In some embodiments, the disclosure provides a kit comprising an aliquot of each of 3 of the following compounds: caprolactam, N-cyclohexylformamide, threo-3-Hydroxy-2-methylbutyric acid, sulfoacetic acid, (2,7-Dimethyloctahydro-1H-cyclopenta[c]pyridin-4-yl)methanol, trans-3-indoleacrylic acid, pantothenic acid, diaveridine, (5-benzyl-3,6-dioxo-2-piperazinyl)acetic acid, dasytrichone, indole, leucoline, skatole, 6-hydroxycaproic acid, 1,2,6-hexanetriol, naphthalen-2-amine, 8-hydroxyquinoline, 2,2,6,6-tetramethyl-4-piperidinol, 1,5-naphthalenediamine, 1H-indole-2-carboxylic acid, (E)-dacarbazine, dinitrosopentamethylenetetramine, 5-hydroxyindole-3-acetic acid, 3-amino-5,7-dimethyl-1-adamantanol, 11-aminoundecanoic acid, kynurenine, N-hydroxy-L-tryptophan, 2-(methylamino)-1H-benzo[de]isoquinoline-1,3(2H)-dione, 5-hydroxy-N-formylkynurenine, DL-tryptophan, phenylalanine, and N-lauroylglycine.

### BRIEF DESCRIPTION OF THE FIGURES

FIG. 1 shows a schematic of protein separation from conditioned media.

FIG. 2 shows a volcano plot of metabolite abundances detected with negative mode or positive mode mass spectrometry.

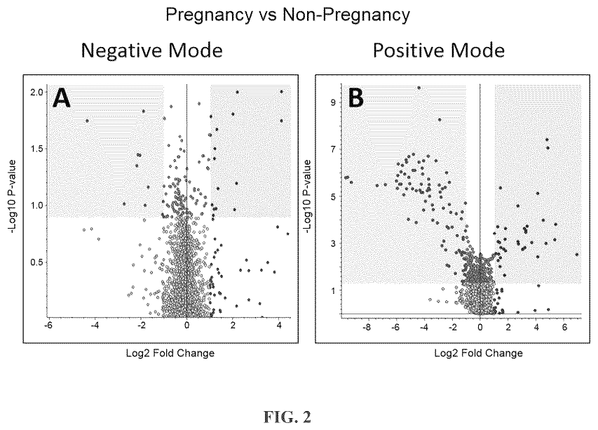

FIG. 3 shows pregnancy indices calculated for samples of conditioned media from embryos that did or did not result in pregnancy following transfer.

### DETAILED DESCRIPTION

Disclosed herein is a method for predicting the implantation success of an embryo during the IVF process. Using a method of the disclosure, one can assess the probability of an embryo's implantation success based on, for example, the concentration of metabolites present in a sample of conditioned embryo culture media.

### In Vitro Fertilization

IVF is a type of ART that involves the fertilization of a female ovum outside of the body. Prior to IVF an ovum must be retrieved from a female subject. Non-limiting examples of female subjects include a human, non-human primate, dog, cow, horse, pig, sheep, goat, cat, buffalo, guinea pig, hamster, rabbit, rat, and mouse. Prior to ovum retrieval, a female's ovaries can be stimulated via the administration of one or more hormones or agents including, for example, inhibin, an inhibin and activin mixture, clomiphene citrate, human menopausal gonadotropins such as follicle stimulating hormone (FSH), and a mixture of FSH and luteinizing hormone (LH), and/or human chorionic gonadotropins.

The retrieval of oocytes can occur through various methods including, for example, transvaginal, ultrasound-guided follicular aspiration, perurethral/transvesical ultrasonographic puncture, or through laparoscopic methods. In some embodiments, immature oocytes can be retrieved and allowed to mature in vitro. In some embodiments, oocytes can be developed from ovarian stem cells, mesenchymal stem cells, or ovarian tissue.

Following retrieval, oocytes are washed and placed in a receptacle such as a dish. After about 2 to 6 hours, eggs are fertilized with sperm via direct injection of sperm into each ovum (intracytoplasmic sperm injection), or by mixing of sperm and oocytes in a dish under conditions that facilitate fertilization. The fertilized oocytes (now embryos) are then cultured in vitro in media for a period of a time, such as 2 to 6 days. Embryo culture conditions can include a temperature approximating that found in vivo (37° C.), sub-ambient concentration of oxygen (usually 5%) and elevated concentrations of carbon dioxide (5-6%). Embryos can be cryopreserved at any time after fertilization occurs.

After the culture period, the embryo transfer process can occur. To facilitate embryo transfer, a speculum can be inserted into the vagina of a subject to open the vaginal walls. A catheter is then passed through the cervix and into the uterine cavity. The optimal placement of the catheter within the uterus is about 1-2 cm from the uterine fundus, and in some instances, catheter placement is guided with ultrasound. Following catheter placement, one or more embryos are passed through the catheter and into the uterus where implantation can occur. Implantation of the embryo into the uterine wall results in pregnancy.

The implantation rate of an embryo following transfer to the uterus is about 30-40% in women under 35 years of age. To improve the efficiency of IVF, clinicians can transfer multiple embryos at once. While transferring multiple embryos at once improves the chances of at least one embryo implanting and thus a successful pregnancy, multiple gestations (i.e., twins, triplets, etc.) can occur. Multiple gestation pregnancies can lead to increased hospital costs, increased rearing burden, and developmental issues. A method disclosed herein can assess the probability of an embryo's implantation success during the embryo culture stage. Knowing the probability of success prior to implantation improves implantation rates and reduces the need for multiple embryo transfers.

### Predicting Embryo Implantation Success

A method of the disclosure can assess the probability of an embryo's implantation success by measuring, for example, the amount of one or more metabolites present in conditioned embryo culture media. Conditioned embryo culture media is culture media that has previously been exposed to an embryo, while fresh culture media is culture media that has not been exposed to an embryo. The presence of metabolites in conditioned embryo culture media can be due to the secretion of the metabolites by the embryo. In some instances, embryos consume or modify metabolites present in fresh embryo culture media causing a change in the amount of the metabolites present in the conditioned embryo culture media. The amount of a specific metabolite present in conditioned embryo culture media can correlate with the probability of implantation success of the cultured embryo. When the amount of a metabolite present in conditioned culture media correlates with a high probability of implantation success, that metabolite suitable as a pregnancy biomarker. When the amount of a metabolite present in conditioned culture media correlates with a low probability of implantation success, the metabolite is suitable as a non-pregnancy biomarker. In some embodiments, a method of the disclosure can predict implantation success of an embryo by comparing the amount of pregnancy vs. non-pregnancy biomarkers present in conditioned culture media.

### Discovery of Pregnancy and Non-Pregnancy Biomarkers

The probing of pregnancy and non-pregnancy biomarkers can be facilitated by, for example, mass spectrometry (MS)-based approaches. MS is an analytical technique that can detect, identify, and quantify molecules, such as metabolites, based on their mass-to-charge (m/z) ratio. The first step in the mass spectrometry process is ionization. Ionization can occur through several different methods. For gaseous or vapor samples, electron ionization or chemical ionization can be used. For solid or liquid biological samples, electrospray ionization (ESI) and matrix-assisted laser desorption/ionization can be used. Other examples of ionization include fast atom bombardment atmospheric-pressure chemical ionization, inductively coupled plasma ionization, photoionization, glow discharge ionization, filed desorption ionization, thermospray ionization, spark ionization, atmospheric pressure chemical ionization, atmospheric pressure photoionization, thermal ionization, and desorption/ionization on silicon. Ionization techniques can be performed that encourage the formation of positive ions (positive mode) or negative ions (negative mode).

Following the ionization of samples, the produced ions are separated according to their m/z ratios. The separation of ions can be performed using several different mass analyzers including, for example, time-of-flight (TOF) analyzers, quadrupole mass analyzers, three-dimensional quadrupole ion traps, cylindrical ion traps, Orbitrap mass analyzers, and Fourier transform ion cyclotron resonance mass analyzers. TOF analyzers use an electric field to accelerate ions through a constant potential and determine the mass of an ion based on the time needed for an ion to reach the detector. In quadrupole mass analyzers, 4 parallel rods create a radio frequency (RF) quadrupole field. Oscillating electric fields are then used to stabilize or destabilize selectively the paths of ions through the RF quadrupole field. At a given time interval, only ions in a certain m/z range pass through the system. Thus, ions are separated.

Three-dimensional quadrupole ion traps function based on the same principles of a quadrupole mass analyzer. However, ions are trapped in a mainly RF-field in a space defined by a ring electrode, and are then sequentially ejected. The ejection of ions is based on the m/z ratio of ions, allowing for separation. Cylindrical ion traps are derivative of quadrupole ion traps, with the difference being that the electrodes are formed from flat rings and are not hyperbolically shaped. Linear ion quadrupole ion traps are similar to quadrupole ion traps, but utilize a two dimensional quadrupole field.

Orbitrap mass analyzers electrostatically trap ions in an orbit around a central, spindle shaped electrode. The electrode traps into oscillation along the electrode's long axis while also orbiting around the electrode. Ion oscillation generates an image current dependent on ion m/z ratio, and can be converted to a mass spectrum by Fourier transformation. Fourier transform ion cyclotron resonance mass analyzers detect the image current produced by ions cyclotroning in the presence of a magnetic field. Ions are injected into a static electric/magnetic ion trap (Penning trap) and effectively form part of a circuit. As ions pass near detectors, electrical signals are measured over time, producing a periodic signal with a frequency dependent on the ion's m/z ratio. Using Fourier transformation, the periodic signal can be deconvolved to obtain a mass spectrum.

Mass spectrometry can be performed in multiple rounds in a process known as tandem mass spectrometry (MS/MS). Rounds of mass spectrometry can be separated by molecule fragmentation. Thus, MS/MS can have the steps of ionization, m/z separation to produce an MS¹ precursor ion, fragmentation of the MS¹ precursor ion, and m/z separation to produce an MS² product ion, followed by detection. Methods for fragmentation include, for example, collision-induced dissociation (CID), higher-energy collisional dissociation (HCD), electron capture dissociation (ECD), electron transfer dissociation (ETD), infrared multiphoton dissociation (IRMPD), blackbody infrared radiative dissociation (BIRD), electron-detachment dissociation (EDD) and surface-induced dissociation (SID).

The ability of MS-based approaches to identify molecules in a complex mixture can be enhanced by combining MS-based approaches with separation techniques. Combining MS approaches with separation techniques allows for the analysis of pre-separated samples. Separation techniques that can be combined with MS-based approaches include, for example, high performance liquid chromatography (HPLC), gas chromatography (GC), ultra-performance liquid chromatography (UPLC), ion chromatography (IC), and capillary electrophoresis (CE).

Non-limiting examples of MS-based approaches that can be used to identify pregnancy or non-pregnancy biomarkers in conditioned media include Fourier transform ion cyclotron resonance-mass spectrometry (FTICR-MS), Fusion Orbitrap MS, UPLC-coupled Fusion Orbitrap MS, collision-induced-dissociation-tandem MS (CID-MS/MS), higher-energy collisional dissociation-coupled tandem MS (HCD-MS/MS), infrared multiphoton dissociation-tandem MS (IRMPD-MS/MS), liquid chromatography triple quadrupole tandem mass spectrometry (LC-QqQ-MS), GC-MS, LC-MS, IC-MS, and capillary electrophoresis-MS (CE-MS).

Completion of MS or MS/MS techniques produces a plot of intensity vs. m/z ratio known as a mass spectrum. The mass spectrum represents the distribution of ions in a sample by m/z ratio. From this distribution the identity of a molecule can be determined. Combining separation techniques with MS approaches allows for separation, analysis, and identification of individual molecules such as pregnancy or non-pregnancy biomarkers from a complex mixture such as conditioned media. In some instances, techniques disclosed herein can identify molecules such as pregnancy or non-pregnancy biomarkers by, for example, molecular weight, chromatographic retention time, mass spectra fragmentation pattern, chemical name, chemical formula, chemical structure, or any combination thereof. Moreover, because the intensity of a molecule's mass spectrum correlates with abundance, MS-based approaches can be used to quantify the amount of a molecule such as a pregnancy or non-pregnancy biomarker present in a sample such as conditioned media.

A metabolite can be identified as a pregnancy of non-pregnancy biomarker if the amount of the metabolite present in embryo conditioned media correlates with a specific pregnancy outcome, such as a successful or non-successful embryo implantation. To determine whether the amount of a metabolite correlates with a pregnancy outcome, statistical methods such as volcano plots can be employed. To produce a volcano plot, the fold change of a measurement of interest between two samples is determined. In some embodiments, the measurement of interest is the abundance of a metabolite in the samples. In some embodiments, the two samples are conditioned media from an embryo resulting in a successful pregnancy and conditioned media from an embryo that did not result in a successful pregnancy. A p-value is also calculated to assess the statistical significance of the change of the measurement seen between the two sample groups. The −log10 (p-value) is then plotted vs. the −log2 (fold change). By setting cut-off values for −log10 (p-value) and −log2 (fold change), biomarkers that correlate with pregnancy or non-pregnancy can be identified.

### Biomarker Based Assessments

A pregnancy or non-pregnancy biomarker can be present in conditioned culture media at any time after fertilization has occurred and embryo culture has begun. In some embodiments, a pregnancy or non-pregnancy biomarker is present in media after about 0.05 days to about 8 days of embryo culture. In some embodiments a pregnancy or non-pregnancy biomarker is present in media after: about 0.05 days to about 0.1 days, about 0.05 days to about 0.2 days, about 0.05 days to about 0.5 days, about 0.05 days to about 1 day, about 0.05 days to about 2 days, about 0.05 days to about 3 days, about 0.05 days to about 4 days, about 0.05 days to about 5 days, about 0.05 days to about 6 days, about 0.05 days to about 7 days, about 0.05 days to about 8 days, about 0.1 days to about 0.2 days, about 0.1 days to about 0.5 days, about 0.1 days to about 1 day, about 0.1 days to about 2 days, about 0.1 days to about 3 days, about 0.1 days to about 4 days, about 0.1 days to about 5 days, about 0.1 days to about 6 days, about 0.1 days to about 7 days, about 0.1 days to about 8 days, about 0.2 days to about 0.5 days, about 0.2 days to about 1 day, about 0.2 days to about 2 days, about 0.2 days to about 3 days, about 0.2 days to about 4 days, about 0.2 days to about 5 days, about 0.2 days to about 6 days, about 0.2 days to about 7 days, about 0.2 days to about 8 days, about 0.5 days to about 1 day, about 0.5 days to about 2 days, about 0.5 days to about 3 days, about 0.5 days to about 4 days, about 0.5 days to about 5 days, about 0.5 days to about 6 days, about 0.5 days to about 7 days, about 0.5 days to about 8 days, about 1 day to about 2 days, about 1 day to about 3 days, about 1 day to about 4 days, about 1 day to about 5 days, about 1 day to about 6 days, about 1 day to about 7 days, about 1 day to about 8 days, about 2 days to about 3 days, about 2 days to about 4 days, about 2 days to about 5 days, about 2 days to about 6 days, about 2 days to about 7 days, about 2 days to about 8 days, about 3 days to about 4 days, about 3 days to about 5 days, about 3 days to about 6 days, about 3 days to about 7 days, about 3 days to about 8 days, about 4 days to about 5 days, about 4 days to about 6 days, about 4 days to about 7 days, about 4 days to about 8 days, about 5 days to about 6 days, about 5 days to about 7 days, about 5 days to about 8 days, about 6 days to about 7 days, about 6 days to about 8 days, or about 7 days to about 8 days of embryo culture. In some embodiments a pregnancy or non-pregnancy biomarker is present in media after: about 0.05 days, about 0.1 days, about 0.2 days, about 0.5 days, about 1 day, about 2 days, about 3 days, about 4 days, about 5 days, about 6 days, about 7 days, or about 8 days of embryo culture. In some embodiments a pregnancy or non-pregnancy biomarker is present in media after at least: about 0.05 days, about 0.1 days, about 0.2 days, about 0.5 days, about 1 day, about 2 days, about 3 days, about 4 days, about 5 days, about 6 days, or about 7 days of embryo culture. In some embodiments a pregnancy or non-pregnancy biomarker is present in media after at most: about 0.1 days, about 0.2 days, about 0.5 days, about 1 day, about 2 days, about 3 days, about 4 days, about 5 days, about 6 days, about 7 days, or about 8 days of embryo culture.

Conditioned media can be collected at any time after culture has begun to determine the presence or amount of a pregnancy or non-pregnancy biomarker in conditioned media. In some embodiments, media is collected after: about 0.05 days to about 8 days of embryo culture. In some embodiments, media is collected after: about 0.05 days to about 0.1 days, about 0.05 days to about 0.2 days, about 0.05 days to about 0.5 days, about 0.05 days to about 1 day, about 0.05 days to about 2 days, about 0.05 days to about 3 days, about 0.05 days to about 4 days, about 0.05 days to about 5 days, about 0.05 days to about 6 days, about 0.05 days to about 7 days, about 0.05 days to about 8 days, about 0.1 days to about 0.2 days, about 0.1 days to about 0.5 days, about 0.1 days to about 1 day, about 0.1 days to about 2 days, about 0.1 days to about 3 days, about 0.1 days to about 4 days, about 0.1 days to about 5 days, about 0.1 days to about 6 days, about 0.1 days to about 7 days, about 0.1 days to about 8 days, about 0.2 days to about 0.5 days, about 0.2 days to about 1 day, about 0.2 days to about 2 days, about 0.2 days to about 3 days, about 0.2 days to about 4 days, about 0.2 days to about 5 days, about 0.2 days to about 6 days, about 0.2 days to about 7 days, about 0.2 days to about 8 days, about 0.5 days to about 1 day, about 0.5 days to about 2 days, about 0.5 days to about 3 days, about 0.5 days to about 4 days, about 0.5 days to about 5 days, about 0.5 days to about 6 days, about 0.5 days to about 7 days, about 0.5 days to about 8 days, about 1 day to about 2 days, about 1 day to about 3 days, about 1 day to about 4 days, about 1 day to about 5 days, about 1 day to about 6 days, about 1 day to about 7 days, about 1 day to about 8 days, about 2 days to about 3 days, about 2 days to about 4 days, about 2 days to about 5 days, about 2 days to about 6 days, about 2 days to about 7 days, about 2 days to about 8 days, about 3 days to about 4 days, about 3 days to about 5 days, about 3 days to about 6 days, about 3 days to about 7 days, about 3 days to about 8 days, about 4 days to about 5 days, about 4 days to about 6 days, about 4 days to about 7 days, about 4 days to about 8 days, about 5 days to about 6 days, about 5 days to about 7 days, about 5 days to about 8 days, about 6 days to about 7 days, about 6 days to about 8 days, or about 7 days to about 8 days of embryo culture. In some embodiments conditioned media is collected after at least: about 0.05 days, about 0.1 days, about 0.2 days, about 0.5 days, about 1 day, about 2 days, about 3 days, about 4 days, about 5 days, about 6 days, or about 7 days of embryo culture. In some embodiments conditioned media is collected: at most about 0.1 days, about 0.2 days, about 0.5 days, about 1 day, about 2 days, about 3 days, about 4 days, about 5 days, about 6 days, about 7 days, or about 8 days of embryo culture.

The probability of an embryo's implantation success can be assessed based on the amount of one or more biomarkers present in conditioned media. A biomarker can be a pregnancy or non-pregnancy biomarker. In some embodiments an embryo's implantation success is based on the amount of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 pregnancy or non-pregnancy biomarkers, or any combination thereof.

In some embodiments, an embryo's probability of implantation success can be assessed based on an amount of about 1 biomarker to about 1,000 biomarkers. In some embodiments, an embryo's implantation success can be assessed based on an amount of about 1 biomarker to about 10 biomarkers, about 1 biomarker to about 50 biomarkers, about 1 biomarker to about 100 biomarkers, about 1 biomarker to about 200 biomarkers, about 1 biomarker to about 500 biomarkers, about 1 biomarker to about 750 biomarkers, about 1 biomarker to about 1,000 biomarkers, about 10 biomarkers to about 50 biomarkers, about 10 biomarkers to about 100 biomarkers, about 10 biomarkers to about 200 biomarkers, about 10 biomarkers to about 500 biomarkers, about 10 biomarkers to about 750 biomarkers, about 10 biomarkers to about 1,000 biomarkers, about 50 biomarkers to about 100 biomarkers, about 50 biomarkers to about 200 biomarkers, about 50 biomarkers to about 500 biomarkers, about 50 biomarkers to about 750 biomarkers, about 50 biomarkers to about 1,000 biomarkers, about 100 biomarkers to about 200 biomarkers, about 100 biomarkers to about 500 biomarkers, about 100 biomarkers to about 750 biomarkers, about 100 biomarkers to about 1,000 biomarkers, about 200 biomarkers to about 500 biomarkers, about 200 biomarkers to about 750 biomarkers, about 200 biomarkers to about 1,000 biomarkers, about 500 biomarkers to about 750 biomarkers, about 500 biomarkers to about 1,000 biomarkers, about 750 biomarkers to about 1,000 biomarkers, about 12 biomarkers to about 35 biomarkers, about 12 biomarkers to about 36 biomarkers, about 20 biomarkers to about 82 biomarkers, or about 20 biomarkers to about 90 biomarkers. In some embodiments, an embryo's implantation success can be assessed based on an amount of about 1 biomarker, about 10 biomarkers, about 50 biomarkers, about 100 biomarkers, about 200 biomarkers, about 500 biomarkers, about 750 biomarkers, or about 1,000 biomarkers. In some embodiments an embryo's implantation success can be assessed based on an amount of at least about 1 biomarker, about 10 biomarkers, about 50 biomarkers, about 100 biomarkers, about 200 biomarkers, about 500 biomarkers, or about 750 biomarkers. In some embodiments, an embryo's implantation success can be assessed based on an amount of at most about 10 biomarkers, about 50 biomarkers, about 100 biomarkers, about 200 biomarkers, about 500 biomarkers, about 750 biomarkers, or about 1,000 biomarkers. A biomarker can be a pregnancy or non-pregnancy biomarker. In some embodiments, an embryo's probability of implantation success can be assessed based on a combination of amounts of pregnancy and non-pregnancy biomarkers.

In some embodiments, an embryo's probability of implantation success can be assessed based on the amount of one or more pregnancy biomarkers present in conditioned media. In some embodiments, an embryo's probability of implantation success can be assessed based on the amount of one or more non-pregnancy biomarkers in conditioned media. In some embodiments, an embryo's probability of implantation success can be assessed based on a comparison of the amount of one or more pregnancy biomarkers to the amount of one or more non-pregnancy biomarkers present in conditioned media. In some embodiments, assessment of an embryo's probability of implantation success is based on a definite amount of one or more pregnancy or non-pregnancy biomarkers. In some embodiments, assessment of an embryo's probability of implantation success is based on a relative amount of one or more pregnancy or non-pregnancy biomarkers.

### Accuracy of Predictions

A method of the disclosure can make a prediction on the implantation success of an embryo. The prediction can be made based on, for example, an assessment of the probability of implantation success of the embryo. The probability of an embryo's implantation success can be based on the presence of pregnancy or non-pregnancy biomarkers in embryo conditioned media. In some embodiments, when predicting whether an embryo will successfully implant, a method of the disclosure has an accuracy of about 70% to about 99.9%. In some embodiments, when predicting whether an embryo will successfully implant, a method of the disclosure has an accuracy of about 70% to about 75%, about 70% to about 80%, about 70% to about 85%, about 70% to about 90%, about 70% to about 95%, about 70% to about 96%, about 70% to about 97%, about 70% to about 98%, about 70% to about 99%, about 70% to about 99.9%, about 75% to about 80%, about 75% to about 85%, about 75% to about 90%, about 75% to about 95%, about 75% to about 96%, about 75% to about 97%, about 75% to about 98%, about 75% to about 99%, about 75% to about 99.9%, about 80% to about 85%, about 80% to about 90%, about 80% to about 95%, about 80% to about 96%, about 80% to about 97%, about 80% to about 98%, about 80% to about 99%, about 80% to about 99.9%, about 85% to about 90%, about 85% to about 95%, about 85% to about 96%, about 85% to about 97%, about 85% to about 98%, about 85% to about 99%, about 85% to about 99.9%, about 90% to about 95%, about 90% to about 96%, about 90% to about 97%, about 90% to about 98%, about 90% to about 99%, about 90% to about 99.9%, about 95% to about 96%, about 95% to about 97%, about 95% to about 98%, about 95% to about 99%, about 95% to about 99.9%, about 96% to about 97%, about 96% to about 98%, about 96% to about 99%, about 96% to about 99.9%, about 97% to about 98%, about 97% to about 99%, about 97% to about 99.9%, about 98% to about 99%, about 98% to about 99.9%, or about 99% to about 99.9%. In some embodiments, when predicting whether an embryo will successfully implant, a method of the disclosure has an accuracy of about 70%, about 75%, about 80%, about 85%, about 90%, about 95%, about 96%, about 97%, about 98%, about 99%, or about 99.9%. In some embodiments, when predicting whether an embryo will successfully implant, a method of the disclosure has accuracy of at least about 70%, at least about 75%, at least about 80%, at least about 85%, at least about 90%, at least about 95%, at least about 96%, at least about 97%, at least about 98%, at least or about 99%.

### Detection Methods

The amount of a metabolite such as a pregnancy or non-pregnancy biomarker present in conditioned culture media can be measured with various detection methods. Non-limiting examples of detection methods that can be used to identify or measure amounts of metabolites include colorimetric assays; optical assays such as fluorescence assays, ultraviolet light (UV) absorbance assays, luminescence assays, and phosphorescence assays; ligand binding assays such as dual polarization interferometry, enzyme-linked immunoabsorbent assays (ELISA), magnetic immunoassays, and radioimmunoassays; spectroscopic assays such as nuclear magnetic resonance (NMR) spectroscopy, Fourier transform infrared (FTIR) spectroscopy, and near infrared spectroscopy (NIR); electrophoresis based assays; nanoparticle-based assays; chromatographic assays such as high performance liquid chromatography (HPLC), gas chromatography (GC), ultra-performance liquid chromatography (UPLC), ion chromatography (IC), capillary electrophoresis (CE) and supercritical fluid chromatography; and MS-based approaches such as FTICR-MS, Orbitrap MS, time of flight-MS (TOF-MS), single quad mass spectroscopy (Q-MS), ion trap mass spectroscopy (IT-MS); tandem based mass spectroscopy such as collision-induced-dissociation-tandem MS (CID-MS/MS), higher-energy collisional dissociation-coupled tandem MS (HCD-MS/MS), infrared multiphoton dissociation-tandem MS (IRMPD-MS/MS); coupled chromatography with mass spectroscopy techniques such as liquid chromatography triple quadrupole tandem mass spectrometry (LC-QqQ-MS), GC-MS, LC-MS, IC-MS, and capillary electrophoresis-MS (CE-MS). In some embodiments, combination of the aforementioned detection methods can be used to measure the amount of one or more metabolites (e.g., pregnancy or non-pregnancy biomarkers) in a sample of conditioned culture media.

The metabolites that are produced, consumed, or modified by an embryo can vary depending on the type of media in which the embryo is cultured. Thus, the classification of a metabolite as a pregnancy or non-pregnancy biomarker can differ depending on an embryo's culture media. Non-limiting examples of media that can be used for embryo culture include SAGE™ In-Vitro Maturation Media (IVM), Vitrolife G-TL™ media, Origio SAGE 1-Step™ media, Cook Medical Sydney IVF Cleavage Medium, Irvine Continuous Single Culture® Complete product (CSCM-C) with Human Serum Albumin, LifeGlobal® Global® Total®, and FertiPro FertiCult™ IVF medium.

### Pregnancy Biomarkers

Non-limiting examples of pregnancy biomarkers that can be utilized in a method of the disclosure are shown below in TABLE 1A and TABLE 1B. TABLE 1A shows details on biomarker chemical formulas, molecular weight, retention time, and MS fragmentation list. TABLE 1B shows corresponding chemical structures of compounds listed in TABLE 1A.

TABLE 1A

| | | | Molecular | RT | Fragmentation |
|---|---|---|---|---|---|
| NO. | Name | Formula | Weight | [min] | list |
| 1 | Caprolactam | C₆H₁₁NO | 113.08393 | 6.483 | 52.9, 54.1, 54.9, |
| | | | | | 56.8, 60.9, 67.0, |
| | | | | | 67.9, 68.9, 70.0, |
| | | | | | 71.0, 72.0, 72.9, |
| | | | | | 73.6, 76.9, 79.0, |
| | | | | | 79.9, 80.9, 83.9, |
| | | | | | 85.1, 85.9, 90.9, |
| | | | | | 95.0, 96.1, 97.0, |
| | | | | | 98.0, 105.0, 114.0 |
| 2 | N-Cyclohexylformamide | C₇H₁₃NO | 127.09952 | 7.616 | 52.8, 54.0, 54.8, |
| | | | | | 55.7, 56.8, 57.9, |
| | | | | | 67.0, 67.8, 69.0, |
| | | | | | 69.8, 70.9, 71.9, |
| | | | | | 74.2, 79.7, 81.9, |
| | | | | | 83.0, 84.1, 86.0, |
| | | | | | 91.4, 98.1, 100.0, |
| | | | | | 101.8, 109.8, |
| | | | | | 111.4, 117.7, |
| | | | | | 128.0 |
| 3 | — | C₉H₆O | 130.04175 | 3.39 | 54.8, 56.8, 62.8, |
| | | | | | 71.9, 73.1, 74.9, |
| | | | | | 76.9, 94.9, 103.0, |
| | | | | | 103.9, 105.0, |
| | | | | | 107.8, 131.0 |
| 4 | — | C₉H₆O | 130.0418 | 3.114 | 50.9, 56.8, 58.8, |
| | | | | | 61.1, 63.0, 66.9, |
| | | | | | 70.0, 71.8, 73.0, |
| | | | | | 76.9, 78.9, 85.1, |
| | | | | | 90.0, 91.0, 94.9, |
| | | | | | 95.6, 100.8, |
| | | | | | 102.1, 102.9, |
| | | | | | 103.7, 104.9, |
| | | | | | 108.0, 118.1, |
| | | | | | 126.1, 130.8 |
| 5 | Threo-3-hydroxy-2- | C₆H₁₂O₃ | 132.07852 | 6.487 | 50.8, 52.8, 56.8, |
| | methylbutyric acid | | | | 66.1, 69.2, 72.9, |
| | | | | | 76.7, 86.8, 87.9, |
| | | | | | 88.8, 95.0, 131.3, |
| | | | | | 133.1 |
| 6 | — | C₄H₁₁O₃P | 138.04421 | 6.238 | 56.7, 58.7, 59.7, |
| | | | | | 62.9, 64.8, 73.0, |
| | | | | | 77.0, 79.8, 81.1, |
| | | | | | 81.9, 93.1, 95.1, |
| | | | | | 97.8, 98.8, 100.9, |
| | | | | | 112.2, 116.0, |
| | | | | | 116.8, 118.8, |
| | | | | | 120.9, 138.8 |
| 7 | Sulfoacetic acid | C₂H₄O₅S | 139.94387 | 0.382 | 52.2, 54.7, 56.7, |
| | | | | | 58.9, 67.1, 67.8, |
| | | | | | 80.9, 81.9, 85.0, |
| | | | | | 85.8, 88.8, 93.1, |
| | | | | | 94.9, 98.9, 100.0, |
| | | | | | 114.8, 115.8, |
| | | | | | 116.9, 117.9, |
| | | | | | 125.8, 130.8, |
| | | | | | 135.8, 140.9 |
| 8 | — | C₆H₁₄O₄ | 150.08909 | 6.383 | 57.0, 57.6, 66.0, |
| | | | | | 68.8, 72.9, 77.9, |
| | | | | | 78.9, 88.8, 103.1, |
| | | | | | 132.8 |
| 9 | Phenylalanine | C₉H₁₁NO₂ | 165.07875 | 3.392 | 50.9, 53.9, 56.8, |
| | | | | | 64.7, 66.9, 67.9, |
| | | | | | 73.9, 77.0, 77.8, |
| | | | | | 79.0, 80.0, 83.8, |
| | | | | | 85.6, 88.1, 91.1, |
| | | | | | 92.0, 93.1, 94.0, |
| | | | | | 95.0, 95.9, 99.1, |
| | | | | | 102.2, 103.0, |
| | | | | | 104.1, 105.0, |
| | | | | | 105.8, 107.0, |
| | | | | | 107.8, 111.9, |
| | | | | | 114.1, 117.4, |
| | | | | | 118.0, 120.0, |
| | | | | | 120.8, 121.7, |
| | | | | | 123.1, 129.0, |
| | | | | | 131.0, 135.2, |
| | | | | | 139.7, 149.0, |
| | | | | | 166.0 |
| 10 | — | — | 174.55215 | 3.813 | 54.7, 55.8, 56.9, |
| | | | | | 66.0, 66.8, 69.0, |
| | | | | | 69.9, 84.9, 86.9, |
| | | | | | 95.3, 96.2, 100.1, |
| | | | | | 108.0, 117.8, |
| | | | | | 123.9, 127.8, |
| | | | | | 130.2, 131.0, |
| | | | | | 146.9, 174.9 |
| 11 | (2,7-Dimethyloctahydro- | C₁₁H₂₁NO | 183.16204 | 7.552 | 56.7, 66.8, 70.9, |
| | 1H-cyclopenta[c]pyridin- | | | | 72.0, 72.9, 74.9, |
| | 4-yl)methanol | | | | 81.0, 82.1, 82.9, |
| | | | | | 87.0, 95.9, 101.8, |
| | | | | | 104.5, 110.8, |
| | | | | | 112.9, 114.8, |
| | | | | | 116.8, 117.9, |
| | | | | | 123.9, 128.0, |
| | | | | | 131.9, 132.8, |
| | | | | | 139.8, 142.1, |
| | | | | | 144.2, 146.9, |
| | | | | | 147.9, 161.0, |
| | | | | | 163.8, 184.2 |
| 12 | — | C₅H₁₀N₆O₂ | 186.08652 | 3.134 | 54.6, 56.3, 56.9, |
| | | | | | 66.9, 67.9, 70.1, |
| | | | | | 72.8, 79.1, 80.8, |
| | | | | | 82.9, 88.0, 89.1, |
| | | | | | 90.8, 95.0, 97.0, |
| | | | | | 99.0, 103.2, |
| | | | | | 107.2, 112.9, |
| | | | | | 113.9, 114.9, |
| | | | | | 118.6, 119.9, |
| | | | | | 133.8, 168.8 |
| 13 | Trans-3-indoleacrylic | C₁₁H₉NO₂ | 187.06307 | 5.766 | 54.8, 56.7, 58.9, |
| | acid | | | | 65.1, 67.9, 70.8, |
| | | | | | 72.9, 73.8, 76.9, |
| | | | | | 78.1, 87.9, 88.9, |
| | | | | | 89.9, 91.0, 95.0, |
| | | | | | 99.0, 101.0, |
| | | | | | 103.0, 103.9, |
| | | | | | 105.0, 106.0, |
| | | | | | 109.1, 113.9, |
| | | | | | 115.0, 116.0, |
| | | | | | 117.0, 118.0, |
| | | | | | 119.1, 120.1, |
| | | | | | 121.1, 127.0, |
| | | | | | 128.0, 129.0, |
| | | | | | 130.1, 132.0, |
| | | | | | 134.0, 134.8, |
| | | | | | 140.1, 141.3, |
| | | | | | 142.0, 143.0, |
| | | | | | 144.0, 145.0, |
| | | | | | 146.1, 146.8, |
| | | | | | 150.1, 155.0, |
| | | | | | 159.9, 164.8, |
| | | | | | 169.2, 170.0, |
| | | | | | 172.8, 176.1, |
| | | | | | 187.9 |
| 14 | DL-Tryptophan | C₁₁H₁₂N₂O₂ | 204.08955 | 5.874 | 56.7, 57.8, 70.9, |
| | | | | | 74.0, 78.9, 91.0, |
| | | | | | 101.9, 105.1, |
| | | | | | 113.8, 115.0, |
| | | | | | 116.0, 117.1, |
| | | | | | 118.0, 119.0, |
| | | | | | 126.9, 128.1, |
| | | | | | 129.1, 129.9, |
| | | | | | 131.3, 132.0, |
| | | | | | 133.0, 134.0, |
| | | | | | 140.4, 142.0, |
| | | | | | 143.0, 144.0, |
| | | | | | 145.1, 146.0, |
| | | | | | 146.9, 154.3, |
| | | | | | 154.9, 159.0, |
| | | | | | 159.9, 170.1, |
| | | | | | 170.7, 187.1, |
| | | | | | 188.0, 199.3, |
| | | | | | 205.1 |
| 15 | — | C₁₂H₂₅NO₂ | 215.18839 | 2.71 | — |
| 16 | Pantothenic acid | C₉H₁₇NO₅ | 219.11024 | 3.812 | 57.0, 59.1, 66.8, |
| | | | | | 67.9, 68.9, 69.9, |
| | | | | | 71.8, 72.9, 81.1, |
| | | | | | 85.0, 90.0, 91.0, |
| | | | | | 93.8, 95.1, 97.9, |
| | | | | | 100.0, 100.8, |
| | | | | | 102.9, 105.0, |
| | | | | | 112.1, 113.0, |
| | | | | | 113.9, 115.9, |
| | | | | | 122.1, 124.1, |
| | | | | | 134.7, 142.2, |
| | | | | | 156.1, 159.8, |
| | | | | | 183.9, 202.0, |
| | | | | | 220.0 |
| 17 | — | — | 219.13875 | 5.396 | — |
| 18 | — | C₁₀H₁₆N₂O₂S | 228.09711 | 2.618 | 52.9, 54.9, 56.8, |
| | | | | | 60.8, 63.8, 68.9, |
| | | | | | 79.0, 80.9, 81.9, |
| | | | | | 87.1, 90.8, 94.8, |
| | | | | | 97.0, 98.9, 101.0, |
| | | | | | 103.1, 105.0, |
| | | | | | 107.1, 109.2, |
| | | | | | 110.8, 112.8, |
| | | | | | 114.6, 116.9, |
| | | | | | 118.9, 120.9, |
| | | | | | 121.8, 123.0, |
| | | | | | 123.9, 124.9, |
| | | | | | 126.9, 128.9, |
| | | | | | 130.9, 133.0, |
| | | | | | 134.0, 134.9, |
| | | | | | 136.9, 138.2, |
| | | | | | 138.9, 139.8, |
| | | | | | 140.8, 142.9, |
| | | | | | 144.9, 145.6, |
| | | | | | 146.9, 148.1, |
| | | | | | 148.9, 150.2, |
| | | | | | 150.9, 152.9, |
| | | | | | 154.2, 154.9, |
| | | | | | 156.2, 156.9, |
| | | | | | 157.8, 158.7, |
| | | | | | 159.8, 161.0, |
| | | | | | 166.0, 166.8, |
| | | | | | 167.6, 168.9, |
| | | | | | 169.5, 171.8, |
| | | | | | 172.9, 173.5, |
| | | | | | 174.7, 177.9, |
| | | | | | 180.9, 191.0, |
| | | | | | 193.2, 196.0, |
| | | | | | 197.1, 199.0, |
| | | | | | 208.9, 211.2, |
| | | | | | 229.0 |
| 19 | — | C₁₀H₂₂O₆ | 238.14117 | 6.555 | 62.8, 68.9, 73.1, |
| | | | | | 75.1, 84.8, 87.0, |
| | | | | | 88.9, 89.6, 90.7, |
| | | | | | 94.0, 96.9, 102.0, |
| | | | | | 107.0, 112.7, |
| | | | | | 117.0, 130.1, |
| | | | | | 130.9, 133.0, |
| | | | | | 147.9, 153.1, |
| | | | | | 156.8, 165.8, |
| | | | | | 166.7, 178.3, |
| | | | | | 179.0, 210.2 |
| 20 | — | C₁₀H₂₂O₆ | 238.14118 | 6.461 | 54.8, 56.6, 57.2, |
| | | | | | 73.0, 87.0, 88.9, |
| | | | | | 102.0, 107.3, |
| | | | | | 130.9, 133.0, |
| | | | | | 133.8, 135.0, |
| | | | | | 147.9, 148.9, |
| | | | | | 150.9, 152.2, |
| | | | | | 156.8, 167.0, |
| | | | | | 188.0 |
| 21 | — | C₁₀H₂₂O₆ | 238.14122 | 6.256 | 54.8, 86.9, 89.0, |
| | | | | | 89.7, 91.5, 107.0, |
| | | | | | 116.7, 130.8, |
| | | | | | 133.0, 133.8, |
| | | | | | 207.8 |
| 22 | — | C₁₀H₂₂O₆ | 238.14123 | 5.999 | 58.8, 61.0, 63.0, |
| | | | | | 82.8, 86.9, 89.0, |
| | | | | | 89.9, 91.0, 96.0, |
| | | | | | 107.0, 116.8, |
| | | | | | 118.1, 119.0, |
| | | | | | 124.0, 132.1, |
| | | | | | 133.0, 133.8, |
| | | | | | 135.0, 135.8, |
| | | | | | 151.1, 165.7, |
| | | | | | 167.7, 209.8 |
| 23 | — | C₈H₁₄N₆O₃ | 242.11265 | 6.785 | — |
| 24 | — | C₁₁H₂₅NO₅ | 251.17303 | 2.62 | 54.9, 59.9, 86.9, |
| | | | | | 97.2, 104.0, |
| | | | | | 129.0, 132.1, |
| | | | | | 134.7, 141.8, |
| | | | | | 156.1, 163.3, |
| | | | | | 165.0 |
| 25 | — | C₄H₅N₂O₅P₃ | 253.94165 | 0.382 | 54.8, 56.9, 90.8, |
| | | | | | 107.1, 112.9, |
| | | | | | 116.7, 118.7, |
| | | | | | 123.2, 130.9, |
| | | | | | 132.1, 133.0, |
| | | | | | 144.9, 148.8, |
| | | | | | 151.5, 152.8, |
| | | | | | 159.9, 169.9, |
| | | | | | 174.7, 176.7, |
| | | | | | 184.0 |
| 26 | — | C₁₇H₁₈S | 254.1125 | 8.055 | 82.8, 94.8, 149.1, |
| | | | | | 183.2, 197.1, |
| | | | | | 255.0 |
| 27 | — | C₁₃H₁₂N₂O₄ | 260.07929 | 3.2 | 56.7, 58.7, 76.7, |
| | | | | | 85.0, 101.0, |
| | | | | | 109.7, 127.9, |
| | | | | | 130.0, 130.9, |
| | | | | | 131.9, 146.1, |
| | | | | | 148.9, 150.9, |
| | | | | | 155.1, 155.9, |
| | | | | | 157.0, 158.0, |
| | | | | | 169.3, 173.0, |
| | | | | | 174.1, 176.2, |
| | | | | | 183.1, 202.1, |
| | | | | | 205.1 |
| 28 | Diaveridine | C₁₃H₁₆N₄O₂ | 260.12319 | 6.219 | 112.9, 128.9, |
| | | | | | 130.9, 140.8, |
| | | | | | 156.3, 158.1, |
| | | | | | 160.8, 168.7, |
| | | | | | 174.0, 261.1 |
| 29 | 5-Benzyl-3,6-dioxo-2- | C₁₃H₁₄N₂O₄ | 262.09492 | 3.36 | 56.9, 63.5, 69.4, |
| | piperazinyl)acetic acid | | | | 71.0, 85.1, 91.1, |
| | | | | | 92.8, 93.9, 101.9, |
| | | | | | 107.9, 117.1, |
| | | | | | 130.0, 132.0, |
| | | | | | 133.1, 135.1, |
| | | | | | 142.1, 145.8, |
| | | | | | 147.1, 149.2, |
| | | | | | 155.8, 156.9, |
| | | | | | 157.9, 159.0, |
| | | | | | 160.0, 165.0, |
| | | | | | 167.8, 170.8, |
| | | | | | 171.9, 175.1, |
| | | | | | 175.9, 181.5, |
| | | | | | 184.9, 185.9, |
| | | | | | 203.0, 204.7, |
| | | | | | 215.1, 220.7, |
| | | | | | 263.0 |
| 30 | — | C₉H₁₄N₆O₄ | 270.10738 | 8.347 | 107.5, 133.0, |
| | | | | | 136.9, 142.9, |
| | | | | | 143.9, 148.1, |
| | | | | | 148.8, 150.0, |
| | | | | | 153.1, 164.0, |
| | | | | | 182.2, 225.1, |
| | | | | | 271.2 |
| 31 | — | — | 283.19887 | 6.462 | 70.9, 73.0, 86.9, |
| | | | | | 88.9, 89.8, 91.1, |
| | | | | | 106.9, 115.1, |
| | | | | | 130.6, 133.0, |
| | | | | | 133.8, 151.0, |
| | | | | | 156.7, 159.1, |
| | | | | | 163.8, 177.1, |
| | | | | | 195.2, 284.1 |
| 32 | — | — | 283.19888 | 6.552 | 72.9, 86.9, 88.9, |
| | | | | | 89.8, 106.1, |
| | | | | | 107.0, 121.2, |
| | | | | | 131.3, 133.0, |
| | | | | | 133.7, 148.9, |
| | | | | | 150.9, 156.3, |
| | | | | | 163.8, 177.1, |
| | | | | | 195.1, 238.8 |
| 33 | — | — | 283.19901 | 6.245 | 52.6, 87.0, 89.0, |
| | | | | | 90.0, 91.1, 133.0, |
| | | | | | 151.0, 177.0, |
| | | | | | 195.1, 239.1, |
| | | | | | 284.0 |
| 34 | — | C₉H₁₆N₆O₅ | 288.11798 | 2.481 | 54.9, 57.0, 88.8, |
| | | | | | 95.0, 98.7, 115.1, |
| | | | | | 123.1, 130.8, |
| | | | | | 133.1, 144.9, |
| | | | | | 154.9, 156.2, |
| | | | | | 158.8, 160.9, |
| | | | | | 162.9, 167.8, |
| | | | | | 171.0, 172.9, |
| | | | | | 179.2, 187.6, |
| | | | | | 197.0, 271.2, |
| | | | | | 289.1 |
| 35 | — | C₁₄H₂₂N₂OS | 288.11805 | 3.294 | 56.9, 67.0, 69.0, |
| | | | | | 71.0, 129.0, |
| | | | | | 131.7, 149.1, |
| | | | | | 152.9, 154.8, |
| | | | | | 156.6, 166.6, |
| | | | | | 168.9, 171.0, |
| | | | | | 172.7, 184.8, |
| | | | | | 288.9 |
| 36 | Dasytrichone | C₁₈H₁₆O₄ | 296.10462 | 3.391 | 57.0, 65.0, 66.8, |
| | | | | | 72.7, 73.9, 75.0, |
| | | | | | 76.9, 79.0, 79.9, |
| | | | | | 91.1, 93.1, 95.0, |
| | | | | | 95.9, 103.1, |
| | | | | | 104.9, 106.0, |
| | | | | | 107.0, 107.9, |
| | | | | | 121.0, 122.0, |
| | | | | | 130.0, 131.0, |
| | | | | | 149.1, 151.0 |
| 37 | — | C₄H₃N₁S₂Cl₂ | 198.9091 | 0.465 | 74.1, 92.0, 93.0, |
| | | | | | 95.9, 98.0, 107.8, |
| | | | | | 109.0, 113.9, |
| | | | | | 115.7, 116.8, |
| | | | | | 121.9, 126.1, |
| | | | | | 135.8, 140.9, |
| | | | | | 141.8, 160.7, |
| | | | | | 161.4, 162.8, |
| | | | | | 180.0, 197.7 |
| 38 | — | C₉H₈S₃ | 211.9788 | 1.979 | 58.7, 75.1, 89.0, |
| | | | | | 91.0, 96.9, 115.1, |
| | | | | | 122.0, 129.1, |
| | | | | | 135.0, 136.9, |
| | | | | | 142.8, 153.0, |
| | | | | | 167.0 |
| 39 | — | C₄H₂N₂O₅S₂ | 237.9354 | 0.465 | — |
| 40 | — | C₁₀H₂₃N₄O₃P₁ | 278.1511 | 21.081 | — |
| 41 | — | C₁₆H₃₀O₄ | 286.2141 | 20.625 | 58.9, 105.0, |
| | | | | | 109.2, 138.9, |
| | | | | | 146.8, 154.9, |
| | | | | | 170.9, 179.4, |
| | | | | | 184.9, 212.9, |
| | | | | | 223.2, 235.2, |
| | | | | | 285.4 |
| 42 | — | C₆H₅N₆O₄S₁P₁ | 287.9829 | 17.412 | 97.1, 102.0, |
| | | | | | 109.0, 113.1, |
| | | | | | 130.8, 136.9, |
| | | | | | 141.0, 143.0, |
| | | | | | 145.8, 154.9, |
| | | | | | 156.9, 160.9, |
| | | | | | 167.7, 178.9, |
| | | | | | 184.3, 190.8, |
| | | | | | 199.1, 201.0, |
| | | | | | 206.9, 215.0, |
| | | | | | 246.8, 255.8, |
| | | | | | 265.4, 286.5 |
| 43 | — | C₃H₃N₂O₅S₃P₁Cl₁ | 308.8630 | 21.566 | — |
| 44 | — | C₂H₃O₄S₂P₃Cl₂ | 317.8076 | 20.962 | 92.9, 112.9, |
| | | | | | 126.9, 135.8, |
| | | | | | 136.8, 140.6, |
| | | | | | 156.5, 168.7, |
| | | | | | 170.6, 180.7, |
| | | | | | 192.7, 229.2 |
| 45 | — | C₁₂H₂₃N₄O₄P₁ | 318.1457 | 20.87 | 59.1, 92.9, 98.9, |
| | | | | | 99.7, 101.0, |
| | | | | | 113.0, 114.9, |
| | | | | | 116.6, 125.0, |
| | | | | | 126.9, 128.2, |
| | | | | | 128.9, 137.0, |
| | | | | | 141.0, 142.0, |
| | | | | | 142.9, 144.5, |
| | | | | | 153.2, 154.0, |
| | | | | | 155.0, 157.0, |
| | | | | | 158.0, 161.9, |
| | | | | | 171.2, 171.9, |
| | | | | | 172.9, 188.1, |
| | | | | | 189.0, 203.0, |
| | | | | | 213.1, 214.5, |
| | | | | | 215.1, 229.1, |
| | | | | | 256.8, 299.2, |
| | | | | | 315.1, 317.3 |
| 46 | — | C₉H₁₃N₂O₇S₁P₁ | 324.0181 | 6.025 | — |
| 47 | — | C₃H₃N₂O₄S₃P₁Cl₂ | 327.8370 | 24.329 | 92.8, 97.3, 103.0, |
| | | | | | 130.6, 136.9, |
| | | | | | 146.9, 157.0, |
| | | | | | 185.5, 190.5, |
| | | | | | 243.2, 266.7 |
| 48 | — | C₈H₁₃N₂O₇S₂P₁ | 343.9902 | 18.101 | — |
| 49 | — | C₉H₁₄O₁₀P₂ | 344.0062 | 19.623 | — |
| 50 | — | C₂₃H₄₇N₁O₅ | 417.3454 | 22.654 | — |
| 51 | — | C₁₈H₁₁N₂O₈S₁P₁ | 445.9974 | 20.088 | — |
| 52 | — | C₇H₃O₁₃S₃P₃ | 483.7949 | 20.923 | — |

TABLE 1B

| NO. | Structure | Name | Formula |
|---|---|---|---|
| 1 | 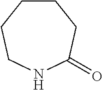 | Caprolactam | C₆H₁₁NO |
| 2 | 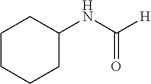 | N-Cyclohexylformamide | C₇H₁₃NO |
| 5 | 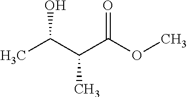 | Threo-3-hydroxy-2- methylbutyric acid | C₆H₁₂O₃ |
| 7 | 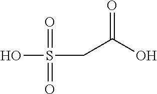 | Sulfoacetic acid | C₂H₄O₅S |
| 9 | 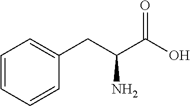 [SMILES: N[C@@H](Cc1ccccc1)C(=O)O] | Phenylalanine | C₉H₁₁NO₂ |
| 11 | 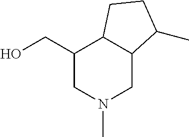 | (2,7-Dimethyloctahydro-1H- cyclopenta[c]pyridin-4- yl)methanol | C₁₁H₂₁NO |
| 13 | 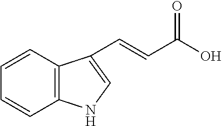 [SMILES: O=C(O)/C=C/c1c[nH]c2ccccc12] | Trans-3-indoleacrylic acid | C₁₁H₉NO₂ |
| 14 | 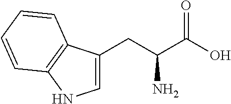 [SMILES: N[C@@H](Cc1c[nH]c2ccccc12)C(=O)O] | DL-Tryptophan | C₁₁H₁₂N₂O₂ |
| 16 | 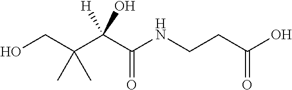 | Pantothenic acid | C₉H₁₇NO₅ |
| 28 | 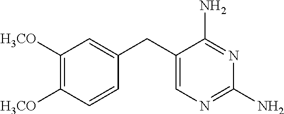 [SMILES: COc1ccc(Cc2cnc(N)nc2N)cc1OC] | Diaveridine | C₁₃H₁₆N₄O₂ |
| 29 | 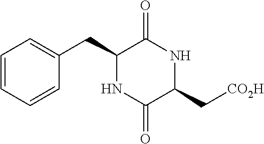 [SMILES: O=C(O)C[C@@H]1NC(=O)[C@H](Cc2ccccc2)NC1=O] | 5-Benzyl-3,6-dioxo-2- piperazinyl)acetic acid | C₁₃H₁₄N₂O₄ |
| 36 | 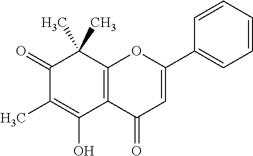 | Dasytrichone | C₁₈H₁₆O₄ |

Non-limiting examples of non-pregnancy biomarkers that can be utilized in a method of the disclosure are shown below in TABLE 2A and TABLE 2B. TABLE 2A shows details on biomarker chemical formulas, molecular weight, time, and MS fragmentation list. TABLE 2B shows corresponding chemical structures of compounds listed in TABLE 1A.

TABLE 2A

| | | | Molecular | RT | Fragmentation |
|---|---|---|---|---|---|
| NO. | Name | Formula | Weight | [min] | list |
| 1 | — | C₆H₁₁NO | 113.08393 | 7.369 | 53.3, 54.2, 54.8, |
| | | | | | 55.9, 56.9, 57.9, |
| | | | | | 58.7, 59.8, 60.8, |
| | | | | | 64.9, 65.7, 66.9, |
| | | | | | 68.0, 69.0, 69.9, |
| | | | | | 70.9, 71.9, 72.8, |
| | | | | | 76.1, 76.8, 79.0, |
| | | | | | 80.8, 81.4, 84.0, |
| | | | | | 86.1, 90.9, 95.0, |
| | | | | | 96.1, 97.0, 105.0, |
| | | | | | 108.6, 111.9, |
| | | | | | 114.0 |
| 2 | Indole | C₈H₇N | 117.0577 | 6.764 | 52.7, 54.8, 55.9, |
| | | | | | 57.1, 57.9, 58.8, |
| | | | | | 59.6, 60.7, 62.0, |
| | | | | | 65.1, 66.9, 67.9, |
| | | | | | 68.7, 70.0, 72.0, |
| | | | | | 72.7, 73.9, 76.1, |
| | | | | | 76.9, 87.0, 88.1, |
| | | | | | 90.1, 90.8, 94.8, |
| | | | | | 100.0, 102.2, |
| | | | | | 105.0, 112.8, |
| | | | | | 117.2, 118.1 |
| 3 | | C₇H₁₃NO | 127.09957 | 8.006 | — |
| 4 | Leucoline | C₉H₇N | 129.05774 | 6.78 | 50.8, 55.8, 60.8, |
| | | | | | 66.8, 76.9, 79.0, |
| | | | | | 84.0, 86.2, 88.0, |
| | | | | | 95.0, 101.0, |
| | | | | | 102.0, 103.0, |
| | | | | | 104.2, 105.0, |
| | | | | | 105.7, 117.9, |
| | | | | | 120.0, 128.0, |
| | | | | | 129.2, 130.1 |
| 5 | Skatole | C₉H₉N | 131.07342 | 6.785 | 57.8, 61.1, 69.0, |
| | | | | | 72.0, 76.9, 78.9, |
| | | | | | 86.0, 90.0, 96.2, |
| | | | | | 97.0, 98.9, 103.8, |
| | | | | | 105.0, 106.2, |
| | | | | | 114.6, 117.0, |
| | | | | | 121.9, 129.9, |
| | | | | | 131.3, 132.0 |
| 6 | 6-Hydroxycaproic acid | C₆H₁₂O₃ | 132.07855 | 7.045 | 56.9, 69.9, 74.0, |
| | | | | | 76.8, 88.0, 91.1, |
| | | | | | 107.7, 122.9, |
| | | | | | 133.2 |
| 7 | 1,2,6-Hexanetriol | C₆H₁₄O₃ | 134.09407 | 5.774 | 52.9, 55.4, 56.1, |
| | | | | | 56.8, 60.8, 68.9, |
| | | | | | 72.9, 74.9, 78.9, |
| | | | | | 80.1, 88.8, 91.0, |
| | | | | | 92.8, 97.0, 105.0, |
| | | | | | 107.0, 116.9, |
| | | | | | 119.0, 122.9, |
| | | | | | 124.9, 129.9, |
| | | | | | 133.0, 135.0 |
| 8 | — | C₄H₁₁O₃P | 138.04419 | 7.038 | 50.9, 52.9, 54.8, |
| | | | | | 56.8, 57.8, 58.8, |
| | | | | | 60.7, 62.8, 64.0, |
| | | | | | 64.9, 66.8, 67.7, |
| | | | | | 68.8, 71.1, 72.9, |
| | | | | | 74.8, 77.0, 77.7, |
| | | | | | 78.8, 79.8, 80.9, |
| | | | | | 82.1, 83.1, 91.0, |
| | | | | | 91.9, 93.1, 93.8, |
| | | | | | 95.0, 97.1, 97.9, |
| | | | | | 98.7, 100.1, |
| | | | | | 101.0, 104.2, |
| | | | | | 105.1, 108.9, |
| | | | | | 111.2, 111.9, |
| | | | | | 114.9, 115.9, |
| | | | | | 116.8, 118.9, |
| | | | | | 121.0, 124.8, |
| | | | | | 136.9, 139.0 |
| 9 | Naphthalen-2-amine | C₁₀H₉N | 143.07328 | 6.749 | 54.7, 56.1, 56.8, |
| | | | | | 57.8, 58.7, 61.9, |
| | | | | | 62.6, 70.0, 70.8, |
| | | | | | 72.9, 74.8, 75.9, |
| | | | | | 76.8, 78.8, 80.1, |
| | | | | | 84.9, 85.9, 86.9, |
| | | | | | 88.9, 90.3, 98.0, |
| | | | | | 100.0, 100.9, |
| | | | | | 101.7, 102.9, |
| | | | | | 103.9, 105.0, |
| | | | | | 108.4, 113.0, |
| | | | | | 115.0, 116.0, |
| | | | | | 116.9, 118.0, |
| | | | | | 118.8, 120.9, |
| | | | | | 121.6, 128.2, |
| | | | | | 128.9, 133.0, |
| | | | | | 134.1, 138.8, |
| | | | | | 140.1, 144.1 |
| 10 | 8-Hydroxyquinoline | C₉H₇NO | 145.05263 | 6.774 | 54.9, 55.9, 57.7, |
| | | | | | 60.0, 63.8, 64.8, |
| | | | | | 72.0, 72.9, 74.0, |
| | | | | | 74.8, 82.2, 86.1, |
| | | | | | 88.1, 90.1, 91.0, |
| | | | | | 94.9, 95.8, 96.8, |
| | | | | | 100.0, 101.0, |
| | | | | | 108.8, 116.9, |
| | | | | | 118.1, 118.8, |
| | | | | | 119.4, 120.9, |
| | | | | | 122.2, 128.0, |
| | | | | | 128.9, 129.8, |
| | | | | | 133.6, 146.1 |
| 11 | — | C₆H₁₄O₄ | 150.0891 | 7.036 | 54.9, 56.8, 64.8, |
| | | | | | 65.9, 68.8, 70.2, |
| | | | | | 73.1, 75.0, 75.5, |
| | | | | | 85.9, 87.0, 87.9, |
| | | | | | 88.9, 104.9, |
| | | | | | 114.9, 118.7, |
| | | | | | 131.8, 132.9 |
| 12 | — | C₄H₈N₆O | 156.076 | 5.79 | 54.9, 56.6, 68.9, |
| | | | | | 69.7, 70.9, 72.7, |
| | | | | | 74.9, 80.9, 90.9, |
| | | | | | 92.9, 94.8, 106.0, |
| | | | | | 111.1, 113.7, |
| | | | | | 115.9, 117.7, |
| | | | | | 119.2, 124.8, |
| | | | | | 131.6, 143.2, |
| | | | | | 157.0 |
| 13 | 2,2,6,6-Tetramethyl-4-piperidinol | C₉H₁₉NO | 157.14657 | 1.955 | 52.7, 54.9, 55.9, |
| | | | | | 56.9, 57.9, 58.8, |
| | | | | | 59.9, 62.0, 64.8, |
| | | | | | 65.9, 66.9, 69.0, |
| | | | | | 69.9, 71.0, 71.8, |
| | | | | | 72.6, 74.0, 74.9, |
| | | | | | 75.8, 77.0, 79.0, |
| | | | | | 81.0, 83.1, 84.1, |
| | | | | | 84.9, 85.9, 86.8, |
| | | | | | 88.0, 91.0, 91.7, |
| | | | | | 93.1, 93.9, 95.1, |
| | | | | | 96.0, 97.0, 98.4, |
| | | | | | 100.1, 102.1, |
| | | | | | 104.8, 108.1, |
| | | | | | 112.0, 112.9, |
| | | | | | 114.1, 115.0, |
| | | | | | 115.9, 116.8, |
| | | | | | 121.8, 123.0, |
| | | | | | 125.0, 130.0, |
| | | | | | 130.9, 138.0, |
| | | | | | 139.9, 140.8, |
| | | | | | 141.9, 143.0, |
| | | | | | 148.8, 157.1, |
| | | | | | 158.0 |
| 14 | 1,5-Naphthalenediamine | C₁₀H₁₀N₂ | 158.08414 | 6.777 | 55.9, 56.8, 61.0, |
| | | | | | 64.9, 65.9, 66.6, |
| | | | | | 71.9, 72.8, 74.8, |
| | | | | | 87.1, 88.9, 89.9, |
| | | | | | 91.0, 97.2, 102.8, |
| | | | | | 107.8, 113.9, |
| | | | | | 115.0, 115.8, |
| | | | | | 116.8, 117.7, |
| | | | | | 126.1, 128.7, |
| | | | | | 129.9, 131.1, |
| | | | | | 132.0, 136.0, |
| | | | | | 140.8, 142.0, |
| | | | | | 146.8, 158.9 |
| 15 | 1H-Indole-2-carboxylic acid | C₉H₇NO₂ | 161.04747 | 6.774 | 52.8, 55.8, 56.8, |
| | | | | | 57.8, 65.9, 67.8, |
| | | | | | 68.9, 70.0, 70.8, |
| | | | | | 71.8, 73.0, 74.8, |
| | | | | | 78.0, 78.9, 80.0, |
| | | | | | 81.8, 83.0, 83.9, |
| | | | | | 84.9, 85.9, 88.0, |
| | | | | | 90.8, 96.9, 97.9, |
| | | | | | 100.1, 100.9, |
| | | | | | 102.0, 103.7, |
| | | | | | 105.0, 106.0, |
| | | | | | 106.9, 108.9, |
| | | | | | 109.8, 113.0, |
| | | | | | 115.9, 118.3, |
| | | | | | 119.0, 120.3, |
| | | | | | 126.9, 129.2, |
| | | | | | 130.8, 138.8, |
| | | | | | 144.0, 145.0, |
| | | | | | 146.2, 147.0, |
| | | | | | 152.8, 155.4, |
| | | | | | 157.2, 162.0 |
| 16 | — | — | 165.07231 | 3.673 | 50.9, 53.7, 64.7, |
| | | | | | 68.1, 72.9, 73.9, |
| | | | | | 75.0, 77.0, 77.7, |
| | | | | | 79.0, 80.0, 84.6, |
| | | | | | 91.0, 92.0, 93.0, |
| | | | | | 94.1, 95.0, 102.4, |
| | | | | | 103.1, 103.9, |
| | | | | | 104.9, 107.0, |
| | | | | | 117.1, 118.0, |
| | | | | | 119.4, 120.0, |
| | | | | | 120.7, 123.9, |
| | | | | | 130.3, 131.0, |
| | | | | | 138.0, 149.2, |
| | | | | | 165.9 |
| 17 | — | C₆H₁₅O₃P | 166.07585 | 3.69 | 54.9, 56.8, 58.8, |
| | | | | | 63.0, 66.9, 72.8, |
| | | | | | 74.9, 77.0, 77.9, |
| | | | | | 78.9, 80.0, 80.9, |
| | | | | | 82.1, 83.0, 84.9, |
| | | | | | 91.0, 92.0, 93.0, |
| | | | | | 94.1, 95.0, 96.0, |
| | | | | | 98.8, 102.3, |
| | | | | | 103.0, 104.1, |
| | | | | | 105.1, 105.9, |
| | | | | | 106.9, 108.0, |
| | | | | | 109.0, 111.2, |
| | | | | | 116.1, 117.1, |
| | | | | | 118.0, 119.1, |
| | | | | | 120.0, 121.0, |
| | | | | | 121.9, 125.0, |
| | | | | | 131.1, 132.2, |
| | | | | | 143.9, 145.0, |
| | | | | | 149.0, 151.1, |
| | | | | | 166.2, 167.0 |
| 18 | — | C₈H₁₆O₄ | 176.10479 | 7.035 | 56.7, 57.7, 66.6, |
| | | | | | 70.8, 72.7, 74.8, |
| | | | | | 79.9, 82.9, 84.9, |
| | | | | | 86.8, 90.9, 92.8, |
| | | | | | 95.0, 98.9, 100.8, |
| | | | | | 103.1, 104.2, |
| | | | | | 110.9, 112.9, |
| | | | | | 114.9, 116.0, |
| | | | | | 116.9, 118.9, |
| | | | | | 121.0, 130.1, |
| | | | | | 131.0, 131.7, |
| | | | | | 132.8, 135.0, |
| | | | | | 135.8, 137.2, |
| | | | | | 139.0, 139.7, |
| | | | | | 148.9, 149.5, |
| | | | | | 165.1, 171.0, |
| | | | | | 177.1 |
| 19 | (E)-Dacarbazine | C₆H₁₀N₆O | 182.09163 | 6.692 | 51.9, 52.7, 54.8, |
| | | | | | 56.9, 63.7, 65.0, |
| | | | | | 67.0, 67.9, 69.7, |
| | | | | | 70.9, 72.9, 78.9, |
| | | | | | 80.8, 81.4, 83.1, |
| | | | | | 84.9, 90.9, 92.9, |
| | | | | | 94.9, 97.1, 97.9, |
| | | | | | 98.9, 99.8, 101.2, |
| | | | | | 105.0, 108.9, |
| | | | | | 113.9, 115.0, |
| | | | | | 116.9, 118.9, |
| | | | | | 121.7, 123.1, |
| | | | | | 124.8, 127.0, |
| | | | | | 128.0, 128.7, |
| | | | | | 130.0, 131.9, |
| | | | | | 133.0, 135.3, |
| | | | | | 137.0, 137.9, |
| | | | | | 139.0, 140.0, |
| | | | | | 141.6, 142.9, |
| | | | | | 144.9, 146.8, |
| | | | | | 152.9, 155.1, |
| | | | | | 156.9, 164.6, |
| | | | | | 165.3, 171.0, |
| | | | | | 181.2, 183.1 |
| 20 | — | C₁₁H₂₁NO | 183.16201 | 8.000 | 54.9, 57.0, 57.8, |
| | | | | | 59.7, 66.9, 68.9, |
| | | | | | 72.0, 78.1, 80.9, |
| | | | | | 82.0, 82.8, 83.9, |
| | | | | | 90.9, 94.4, 95.2, |
| | | | | | 97.8, 99.1, 99.7, |
| | | | | | 110.4, 113.0, |
| | | | | | 113.7, 115.1, |
| | | | | | 116.2, 117.0, |
| | | | | | 117.9, 118.9, |
| | | | | | 128.0, 129.0, |
| | | | | | 130.8, 132.9, |
| | | | | | 139.7, 142.9, |
| | | | | | 143.7, 146.8, |
| | | | | | 148.8, 149.9, |
| | | | | | 153.7, 155.7, |
| | | | | | 172.0, 183.9 |
| 21 | Dinitrosopentamethylenetetramine | C₅H₁₀N₆O₂ | 186.08651 | 2.892 | 56.8, 60.8, 67.0, |
| | | | | | 70.9, 72.8, 74.8, |
| | | | | | 83.0, 87.9, 90.1, |
| | | | | | 90.8, 92.9, 94.8, |
| | | | | | 100.0, 108.9, |
| | | | | | 112.9, 113.9, |
| | | | | | 114.8, 118.9, |
| | | | | | 129.0, 130.9, |
| | | | | | 132.0, 133.9, |
| | | | | | 142.0, 148.0, |
| | | | | | 159.8, 162.9 |
| 22 | — | C₁₁H₉NO₂ | 187.06305 | 6.768 | 54.4, 55.9, 56.9, |
| | | | | | 64.9, 65.8, 70.0, |
| | | | | | 70.8, 72.9, 73.5, |
| | | | | | 74.9, 76.9, 80.8, |
| | | | | | 83.1, 85.7, 88.1, |
| | | | | | 88.9, 90.1, 91.0, |
| | | | | | 95.1, 95.9, 97.1, |
| | | | | | 98.9, 100.8, |
| | | | | | 102.9, 103.8, |
| | | | | | 104.7, 108.9, |
| | | | | | 111.0, 112.1, |
| | | | | | 113.9, 115.0, |
| | | | | | 116.0, 117.0, |
| | | | | | 118.0, 118.9, |
| | | | | | 119.9, 127.0, |
| | | | | | 127.9, 128.8, |
| | | | | | 129.9, 130.9, |
| | | | | | 132.0, 132.9, |
| | | | | | 133.9, 134.9, |
| | | | | | 136.6, 140.0, |
| | | | | | 142.0, 143.0, |
| | | | | | 144.0, 145.0, |
| | | | | | 146.0, 146.9, |
| | | | | | 147.6, 148.9, |
| | | | | | 150.7, 154.9, |
| | | | | | 156.6, 159.9, |
| | | | | | 160.9, 162.8, |
| | | | | | 164.9, 165.6, |
| | | | | | 169.9, 170.7, |
| | | | | | 188.0 |
| 23 | 5-Hydroxyindole-3-acetic acid | C₁₀H₉NO₃ | 191.05794 | 6.774 | 55.8, 56.7, 57.6, |
| | | | | | 64.9, 65.9, 68.8, |
| | | | | | 69.7, 71.0, 72.8, |
| | | | | | 74.0, 74.8, 77.1, |
| | | | | | 80.8, 82.2, 86.9, |
| | | | | | 87.9, 88.9, 90.9, |
| | | | | | 91.8, 93.1, 93.8, |
| | | | | | 98.8, |
| | | | | | 100.0, 100.8, |
| | | | | | 102.9, 103.7, |
| | | | | | 104.9, 106.1, |
| | | | | | 106.9, 108.9, |
| | | | | | 114.9, 115.9, |
| | | | | | 116.8, 117.9, |
| | | | | | 118.8, 119.9, |
| | | | | | 121.0, 129.9, |
| | | | | | 131.1, 131.9, |
| | | | | | 132.8, 134.0, |
| | | | | | 134.8, 136.3, |
| | | | | | 136.9, 141.0, |
| | | | | | 146.0, 146.9, |
| | | | | | 148.9, 149.9, |
| | | | | | 151.0, 151.9, |
| | | | | | 153.1, 160.8, |
| | | | | | 161.9, 164.0, |
| | | | | | 167.2, 168.9, |
| | | | | | 178.8, 187.0, |
| | | | | | 192.0 |
| 24 | — | C₈H₁₈O₅ | 194.11523 | 7.046 | 52.8, 56.9, 59.0, |
| | | | | | 71.0, 72.9, 74.9, |
| | | | | | 76.9, 78.9, 79.9, |
| | | | | | 81.3, 85.1, 89.0, |
| | | | | | 89.9, 90.9, 91.9, |
| | | | | | 93.0, 93.9, 95.2, |
| | | | | | 97.9, 98.8, 100.8, |
| | | | | | 104.0, 105.0, |
| | | | | | 106.1, 107.1, |
| | | | | | 108.2, 109.0, |
| | | | | | 111.0, 112.2, |
| | | | | | 113.0, 114.8, |
| | | | | | 115.9, 116.8, |
| | | | | | 119.1, 121.1, |
| | | | | | 123.8, 128.8, |
| | | | | | 131.1, 134.2, |
| | | | | | 135.1, 136.0, |
| | | | | | 137.0, 138.0, |
| | | | | | 138.9, 141.1, |
| | | | | | 141.9, 146.7, |
| | | | | | 148.8, 150.8, |
| | | | | | 151.9, 153.1, |
| | | | | | 153.8, 163.2, |
| | | | | | 164.9, 166.8, |
| | | | | | 181.0, 182.8, |
| | | | | | 195.0 |
| 25 | 3-Amino-5,7-dimethyl-1- | C₁₂H₂₁NO | 195.16204 | 7.28 | 57.8, 58.5, 59.2, |
| | adamantanol | | | | 60.0, 63.8, 69.0, |
| | | | | | 72.9, 77.2, 78.9, |
| | | | | | 79.9, 80.9, 81.9, |
| | | | | | 85.0, 85.9, 93.2, |
| | | | | | 94.2, 95.1, 98.9, |
| | | | | | 104.8, 106.9, |
| | | | | | 109.0, 114.9, |
| | | | | | 115.6, 117.0, |
| | | | | | 119.2, 119.9, |
| | | | | | 121.4, 125.0, |
| | | | | | 126.8, 130.8, |
| | | | | | 132.0, 134.9, |
| | | | | | 136.8, 138.0, |
| | | | | | 139.2, 140.0, |
| | | | | | 142.0, 142.9, |
| | | | | | 144.8, 149.9, |
| | | | | | 152.8, 154.8, |
| | | | | | 155.6, 158.8, |
| | | | | | 159.7, 165.9, |
| | | | | | 167.0, 167.9, |
| | | | | | 173.0, 196.2 |
| 26 | — | C₈H₁₉N₅O | 201.15921 | 1.958 | 52.9, 53.6, 54.9, |
| | | | | | 55.8, 56.9, 57.9, |
| | | | | | 58.9, 60.0, 61.9, |
| | | | | | 62.7, 65.1, 66.9, |
| | | | | | 67.7, 68.9, 70.0, |
| | | | | | 70.9, 71.9, 79.0, |
| | | | | | 81.0, 81.9, 83.0, |
| | | | | | 84.0, 85.0, 85.6, |
| | | | | | 88.1, 89.9, 92.8, |
| | | | | | 95.0, 95.8, 97.1, |
| | | | | | 99.9, 102.0, |
| | | | | | 103.0, 108.2, |
| | | | | | 111.0, 114.1, |
| | | | | | 116.1, 121.3, |
| | | | | | 123.1, 128.0, |
| | | | | | 141.1, 142.0, |
| | | | | | 146.0, 154.1, |
| | | | | | 183.9, 201.1, |
| | | | | | 202.2, 202.8 |
| 27 | 11-Aminoundecanoic acid | C₁₁H₂₃NO₂ | 201.17262 | 10.058 | 56.9, 58.0, 61.9, |
| | | | | | 83.0, 85.9, 88.0, |
| | | | | | 99.0, 100.1, |
| | | | | | 102.1, 111.1, |
| | | | | | 114.8, 117.0, |
| | | | | | 124.7, 130.0, |
| | | | | | 132.9, 142.1, |
| | | | | | 145.9, 149.9, |
| | | | | | 160.8, 171.9, |
| | | | | | 183.9, 202.1 |
| 28 | — | — | 201.18091 | 1.959 | 54.9, 55.8, 56.9, |
| | | | | | 57.9, 58.9, 59.8, |
| | | | | | 61.9, 62.8, 64.2, |
| | | | | | 65.0, 66.9, 69.1, |
| | | | | | 69.9, 70.9, 71.9, |
| | | | | | 79.0, 81.0, 81.9, |
| | | | | | 83.0, 84.0, 84.9, |
| | | | | | 87.9, 90.0, 94.9, |
| | | | | | 97.1, 97.9, 102.1, |
| | | | | | 103.1, 110.1, |
| | | | | | 113.9, 116.8, |
| | | | | | 121.0, 123.1, |
| | | | | | 128.1, 139.7, |
| | | | | | 140.9, 142.0, |
| | | | | | 145.3, 146.1, |
| | | | | | 183.9, 202.1, |
| | | | | | 202.9 |
| 29 | — | — | 201.18741 | 1.943 | 52.9, 53.6, 54.9, |
| | | | | | 55.8, 56.9, 57.9, |
| | | | | | 58.9, 60.0, 61.9, |
| | | | | | 62.7, 65.1, 66.9, |
| | | | | | 67.7, 68.9, 70.0, |
| | | | | | 70.9, 71.9, 79.0, |
| | | | | | 81.0, 81.9, 83.0, |
| | | | | | 84.0, 85.0, 85.6, |
| | | | | | 88.1, 89.9, 92.8, |
| | | | | | 95.0, 95.8, 97.1, |
| | | | | | 99.9, 102.0, |
| | | | | | 103.0, 108.2, |
| | | | | | 111.0, 114.1, |
| | | | | | 116.1, 121.3, |
| | | | | | 123.1, 128.0, |
| | | | | | 141.1, 142.0, |
| | | | | | 146.0, 154.1, |
| | | | | | 183.9, 201.1, |
| | | | | | 202.2, 202.8 |
| 30 | — | C₁₁H₁₂N₂O₂ | 204.08954 | 6.769 | 54.9, 64.9, 73.9, |
| | | | | | 78.0, 78.9, 81.0, |
| | | | | | 91.0, 91.8, 94.7, |
| | | | | | 102.8, 103.9, |
| | | | | | 105.1, 107.1, |
| | | | | | 109.2, 109.9, |
| | | | | | 115.0, 116.0, |
| | | | | | 117.0, 118.0, |
| | | | | | 118.9, 120.1, |
| | | | | | 121.2, 126.9, |
| | | | | | 127.9, 129.0, |
| | | | | | 130.0, 130.9, |
| | | | | | 132.0, 133.0, |
| | | | | | 134.0, 134.9, |
| | | | | | 136.5, 140.2, |
| | | | | | 141.4, 142.0, |
| | | | | | 143.0, 144.0, |
| | | | | | 145.1, 146.0, |
| | | | | | 146.6, 147.8, |
| | | | | | 148.9, 153.5, |
| | | | | | 154.9, 158.0, |
| | | | | | 159.0, 160.0, |
| | | | | | 160.6, 169.0, |
| | | | | | 170.0, 170.7, |
| | | | | | 177.0, 188.0, |
| | | | | | 190.2, 205.1 |
| 31 | Kynurenine | C₁₀H₁₂N₂O₃ | 208.08442 | 6.787 | 56.9, 58.8, 60.8, |
| | | | | | 67.0, 69.0, 69.9, |
| | | | | | 70.8, 73.8, 78.9, |
| | | | | | 85.0, 91.0, 92.0, |
| | | | | | 94.0, 95.1, 97.0, |
| | | | | | 99.0, 103.9, |
| | | | | | 105.0, 105.7, |
| | | | | | 114.9, 117.0, |
| | | | | | 118.1, 118.8, |
| | | | | | 120.0, 121.0, |
| | | | | | 122.0, 122.9, |
| | | | | | 124.9, 126.7, |
| | | | | | 128.6, 130.0, |
| | | | | | 131.3, 132.0, |
| | | | | | 132.9, 134.8, |
| | | | | | 136.0, 137.0, |
| | | | | | 142.9, 145.0, |
| | | | | | 146.0, 146.9, |
| | | | | | 149.0, 149.9, |
| | | | | | 152.9, 157.8, |
| | | | | | 161.0, 162.0, |
| | | | | | 165.1, 166.1, |
| | | | | | 167.9, 171.3, |
| | | | | | 173.0, 173.9, |
| | | | | | 208.9 |
| 32 | — | C₈H₇N₃O₄ | 209.04265 | 3.603 | — |
| 33 | — | C₇H₁₀N₆O₂ | 210.08653 | 7.637 | — |
| 34 | — | C₆H₁₀N₆O₃ | 214.08142 | 2.899 | 50.8, 56.8, 66.9, |
| | | | | | 68.8, 69.7, 70.9, |
| | | | | | 74.1, 76.7, 77.8, |
| | | | | | 78.9, 80.9, 83.3, |
| | | | | | 84.8, 90.9, 92.9, |
| | | | | | 94.9, 95.9, 104.9, |
| | | | | | 105.9, 107.0, |
| | | | | | 111.0, 115.0, |
| | | | | | 116.1, 116.8, |
| | | | | | 120.9, 123.3, |
| | | | | | 133.0, 142.0, |
| | | | | | 157.8, 159.3, |
| | | | | | 168.8, 215.1 |
| 35 | — | C₁₂H₂₅NO₂ | 215.18832 | 2.988 | 57.8, 58.7, 70.1, |
| | | | | | 72.7, 76.0, 77.3, |
| | | | | | 81.0, 84.1, 85.0, |
| | | | | | 92.8, 95.0, 100.4, |
| | | | | | 102.2, 112.9, |
| | | | | | 116.1, 122.1, |
| | | | | | 123.0, 128.1, |
| | | | | | 142.8, 146.9, |
| | | | | | 157.0, 159.9, |
| | | | | | 172.0, 174.1, |
| | | | | | 206.8 |
| 36 | — | C₁₁H₂₃NO₃ | 217.16748 | 2.638 | — |
| 37 | — | C₁₁H₂₂ClNO | 219.13869 | 5.155 | — |
| 38 | N-Hydroxy-L-tryptophan | C₁₁H₁₂N₂O₃ | 220.08447 | 6.753 | 56.8, 73.9, 81.1, |
| | | | | | 92.8, 93.7, 103.7, |
| | | | | | 104.9, 109.0, |
| | | | | | 111.0, 117.9, |
| | | | | | 120.8, 123.6, |
| | | | | | 127.9, 130.0, |
| | | | | | 131.9, 133.8, |
| | | | | | 135.7, 137.2, |
| | | | | | 145.9, 147.0, |
| | | | | | 148.0, 156.1, |
| | | | | | 156.9, 158.0, |
| | | | | | 163.3, 175.0, |
| | | | | | 176.3, 177.3, |
| | | | | | 178.0, 203.0 |
| 39 | 2-(Methylamino)-1H- | C₁₃H₁₀N₂O₂ | 226.07154 | 6.764 | 54.8, 56.9, 67.1, |
| | benzo[de]isoquinoline-1,3(2H)- | | | | 69.0, 70.7, 71.7, |
| | dione | | | | 78.8, 81.2, 83.0, |
| | | | | | 91.3, 92.5, 93.1, |
| | | | | | 94.8, 95.7, 97.0, |
| | | | | | 99.9, 100.8, |
| | | | | | 107.0, 109.1, |
| | | | | | 110.0, 110.9, |
| | | | | | 111.9, 113.4, |
| | | | | | 115.0, 116.0, |
| | | | | | 121.1, 122.7, |
| | | | | | 128.4, 131.2, |
| | | | | | 134.2, 137.0, |
| | | | | | 138.9, 140.9, |
| | | | | | 143.2, 148.9, |
| | | | | | 150.9, 152.9, |
| | | | | | 154.7, 157.0, |
| | | | | | 164.9, 167.0, |
| | | | | | 168.0, 169.9, |
| | | | | | 171.0, 173.7, |
| | | | | | 178.9, 181.7, |
| | | | | | 183.0, 184.2, |
| | | | | | 185.2, 191.3, |
| | | | | | 197.0, 226.9 |
| 40 | — | C₆H₈N₆O₄ | 228.06053 | 4.957 | 51.7, 52.9, 56.8, |
| | | | | | 80.9, 98.9, 105.0, |
| | | | | | 106.8, 108.8, |
| | | | | | 111.2, 114.9, |
| | | | | | 116.9, 118.2, |
| | | | | | 118.9, 120.8, |
| | | | | | 122.0, 122.9, |
| | | | | | 124.9, 129.1, |
| | | | | | 130.0, 130.9, |
| | | | | | 133.0, 135.0, |
| | | | | | 136.9, 137.6, |
| | | | | | 139.0, 140.9, |
| | | | | | 142.9, 143.7, |
| | | | | | 144.4, 147.0, |
| | | | | | 148.9, 150.8, |
| | | | | | 151.5, 152.9, |
| | | | | | 154.1, 154.9, |
| | | | | | 155.6, 156.9, |
| | | | | | 157.9, 158.8, |
| | | | | | 160.9, 162.7, |
| | | | | | 166.0, 167.2, |
| | | | | | 168.1, 168.9, |
| | | | | | 169.7, 170.6, |
| | | | | | 172.0, 173.0, |
| | | | | | 175.7, 179.0, |
| | | | | | 180.9, 191.1, |
| | | | | | 199.0, 208.9, |
| | | | | | 229.2 |
| 41 | — | C₁₀H₁₆N₂O₂S | 228.09711 | 7.593 | 53.0, 56.7, 70.3, |
| | | | | | 78.9, 80.9, 81.8, |
| | | | | | 90.8, 94.1, 95.0, |
| | | | | | 97.0, 97.9, 98.9, |
| | | | | | 101.0, 104.9, |
| | | | | | 106.8, 111.0, |
| | | | | | 113.9, 114.7, |
| | | | | | 115.9, 116.9, |
| | | | | | 118.0, 118.9, |
| | | | | | 121.1, 123.0, |
| | | | | | 124.9, 126.7, |
| | | | | | 128.8, 129.8, |
| | | | | | 130.9, 132.9, |
| | | | | | 134.9, 136.8, |
| | | | | | 138.9, 139.9, |
| | | | | | 141.0, 142.0, |
| | | | | | 142.9, 143.6, |
| | | | | | 145.0, 146.1, |
| | | | | | 147.1, 148.9, |
| | | | | | 150.9, 153.0, |
| | | | | | 154.1, 154.9, |
| | | | | | 155.8, 156.9, |
| | | | | | 157.6, 159.1, |
| | | | | | 159.9, 161.0, |
| | | | | | 162.8, 168.9, |
| | | | | | 169.5, 172.9, |
| | | | | | 174.9, 175.9, |
| | | | | | 178.9, 181.0, |
| | | | | | 186.9, 187.9, |
| | | | | | 198.1, 198.8, |
| | | | | | 199.5, 209.1, |
| | | | | | 210.9 |
| 42 | — | C₇H₁₄N₆O₃ | 230.11263 | 7.935 | 54.9, 56.9, 69.0, |
| | | | | | 79.1, 82.2, 82.8, |
| | | | | | 95.0, 96.7, 99.0, |
| | | | | | 101.0, 104.1, |
| | | | | | 112.6, 113.9, |
| | | | | | 117.0, 118.9, |
| | | | | | 122.1, 125.8, |
| | | | | | 127.7, 129.0, |
| | | | | | 130.3, 131.6, |
| | | | | | 132.9, 135.0, |
| | | | | | 138.6, 140.7, |
| | | | | | 143.0, 144.1, |
| | | | | | 145.0, 145.6, |
| | | | | | 152.6, 155.1, |
| | | | | | 156.2, 156.8, |
| | | | | | 158.1, 158.9, |
| | | | | | 160.9, 169.0, |
| | | | | | 170.2, 170.9, |
| | | | | | 173.0, 174.9, |
| | | | | | 181.0, 182.9, |
| | | | | | 200.9 |
| 43 | — | C₆H₁₂N₆O₄ | 232.09199 | 2.88 | 56.8, 66.9, 68.9, |
| | | | | | 71.4, 74.0, 76.9, |
| | | | | | 78.0, 83.0, 85.1, |
| | | | | | 92.9, 95.2, 96.2, |
| | | | | | 98.0, 105.0, |
| | | | | | 106.1, 109.0, |
| | | | | | 113.9, 115.7, |
| | | | | | 123.3, 132.9, |
| | | | | | 135.0, 140.8, |
| | | | | | 141.9, 146.1, |
| | | | | | 185.2, 197.9, |
| | | | | | 215.2 |
| 44 | — | — | 232.80632 | 7.033 | — |
| 45 | — | C₅H₁₃N₆O₃P | 236.07933 | 6.767 | 56.8, 67.2, 70.9, |
| | | | | | 72.7, 74.0, 74.9, |
| | | | | | 83.1, 91.1, 92.2, |
| | | | | | 93.2, 93.8, 94.9, |
| | | | | | 97.3, 97.9, 99.0, |
| | | | | | 101.9, 104.8, |
| | | | | | 109.0, 112.9, |
| | | | | | 114.9, 118.0, |
| | | | | | 119.6, 128.0, |
| | | | | | 130.0, 130.9, |
| | | | | | 131.9, 135.8, |
| | | | | | 139.2, 142.1, |
| | | | | | 143.9, 145.1, |
| | | | | | 146.0, 146.8, |
| | | | | | 147.9, 149.0, |
| | | | | | 153.2, 155.9, |
| | | | | | 157.0, 158.8, |
| | | | | | 162.9, 164.0, |
| | | | | | 165.0, 166.7, |
| | | | | | 170.0, 174.0, |
| | | | | | 176.9, 177.9, |
| | | | | | 184.9, 190.9, |
| | | | | | 200.1, 237.0 |
| 46 | — | C₁₀H₂₂O₆ | 238.14122 | 7.044 | 52.8, 56.9, 58.8, |
| | | | | | 62.8, 68.9, 71.0, |
| | | | | | 73.0, 73.9, 80.9, |
| | | | | | 84.1, 84.9, 87.0, |
| | | | | | 88.9, 89.8, 90.9, |
| | | | | | 102.0, 105.0, |
| | | | | | 107.0, 113.0, |
| | | | | | 116.2, 117.3, |
| | | | | | 118.0, 119.0, |
| | | | | | 129.7, 130.9, |
| | | | | | 132.3, 133.0, |
| | | | | | 133.9, 134.9, |
| | | | | | 143.7, 145.8, |
| | | | | | 147.7, 148.8, |
| | | | | | 152.8, 154.5, |
| | | | | | 162.8, 165.0, |
| | | | | | 175.8, 177.0, |
| | | | | | 194.8 |
| 47 | — | — | 238.14871 | 7.023 | 57.0, 59.1, 61.0, |
| | | | | | 65.3, 68.8, 73.0, |
| | | | | | 87.0, 88.9, 89.8, |
| | | | | | 93.1, 103.1, |
| | | | | | 107.0, 109.3, |
| | | | | | 112.0, 112.7, |
| | | | | | 115.9, 117.1, |
| | | | | | 130.9, 133.0, |
| | | | | | 133.8, 134.8, |
| | | | | | 148.9, 153.2, |
| | | | | | 165.8, 176.9, |
| | | | | | 178.0, 195.1 |
| 48 | — | — | 238.14992 | 7.028 | 59.0, 60.9, 72.9, |
| | | | | | 83.7, 87.1, 88.2, |
| | | | | | 89.0, 90.0, 91.0, |
| | | | | | 91.9, 102.9, |
| | | | | | 108.8, 112.8, |
| | | | | | 116.1, 117.1, |
| | | | | | 118.0, 129.9, |
| | | | | | 130.7, 131.5, |
| | | | | | 132.9, 134.0, |
| | | | | | 135.1, 145.9, |
| | | | | | 148.9, 151.0, |
| | | | | | 152.6, 161.1, |
| | | | | | 163.0, 164.7, |
| | | | | | 165.8, 167.0, |
| | | | | | 167.8, 170.4, |
| | | | | | 176.8 |
| 49 | — | C₉H₂₂N₂O₅ | 238.15322 | 6.984 | 57.0, 59.1, 61.0, |
| | | | | | 65.3, 68.8, 73.0, |
| | | | | | 87.0, 88.9, 89.8, |
| | | | | | 93.1, 103.1, |
| | | | | | 107.0, 109.3, |
| | | | | | 112.0, 112.7, |
| | | | | | 115.9, 117.1, |
| | | | | | 130.9, 133.0, |
| | | | | | 133.8, 134.8, |
| | | | | | 148.9, 153.2, |
| | | | | | 165.8, 176.9, |
| | | | | | 178.0, 195.1 |
| 50 | — | — | 239.14699 | 6.919 | 73.1, 73.7, 83.9, |
| | | | | | 87.9, 89.0, 90.0, |
| | | | | | 117.8, 122.1, |
| | | | | | 130.1, 132.0, |
| | | | | | 132.8, 134.0, |
| | | | | | 147.8, 150.3, |
| | | | | | 155.9, 170.2, |
| | | | | | 177.9, 182.0 |
| 51 | — | C₈H₁₂N₆O₃ | 240.09696 | 3.216 | — |
| 52 | — | C₁₂H₂₀N₂O₃ | 240.14766 | 7.145 | 57.8, 80.9, 88.2, |
| | | | | | 89.1, 90.9, 103.2, |
| | | | | | 107.9, 114.1, |
| | | | | | 116.8, 118.9, |
| | | | | | 119.7, 137.0, |
| | | | | | 151.9, 165.1, |
| | | | | | 181.4, 183.0, |
| | | | | | 191.2, 205.1, |
| | | | | | 224.3 |
| 53 | — | C₆H₁₄N₂O₈ | 242.07622 | 3.359 | — |
| 54 | — | C₈H₁₄N₆O₃ | 242.11243 | 7.367 | — |
| 55 | — | C₉H₁₂N₆O₄ | 244.092 | 4.402 | 56.9, 78.9, 94.9, |
| | | | | | 97.9, 105.9, |
| | | | | | 117.0, 131.9, |
| | | | | | 141.8, 144.8, |
| | | | | | 148.7, 152.9, |
| | | | | | 155.8, 164.7, |
| | | | | | 170.9, 179.7, |
| | | | | | 188.6, 196.9 |
| 56 | 5-Hydroxy-N-formylkynurenine | C₁₁H₁₂N₂O₅ | 252.07419 | 6.767 | 56.8, 58.8, 66.8, |
| | | | | | 68.9, 70.9, 77.1, |
| | | | | | 79.3, 80.9, 82.8, |
| | | | | | 85.0, 85.8, 87.0, |
| | | | | | 88.9, 95.0, 96.9, |
| | | | | | 99.0, 101.8, |
| | | | | | 103.0, 103.7, |
| | | | | | 105.1, 107.0, |
| | | | | | 109.0, 113.0, |
| | | | | | 115.0, 116.1, |
| | | | | | 117.0, 118.9, |
| | | | | | 119.6, 120.9, |
| | | | | | 129.0, 131.0, |
| | | | | | 133.0, 133.6, |
| | | | | | 134.9, 137.0, |
| | | | | | 137.7, 140.9, |
| | | | | | 143.0, 145.4, |
| | | | | | 146.7, 149.0, |
| | | | | | 150.1, 150.9, |
| | | | | | 151.9, 153.0, |
| | | | | | 155.0, 156.1, |
| | | | | | 158.2, 159.0, |
| | | | | | 160.3, 160.9, |
| | | | | | 161.6, 164.8, |
| | | | | | 167.3, 170.9, |
| | | | | | 175.3, 176.8, |
| | | | | | 178.7, 186.9, |
| | | | | | 189.0, 194.6, |
| | | | | | 206.9, 253.0 |
| 57 | — | C₁₀H₂₀O₇ | 252.12046 | 7.31 | — |
| 58 | — | C₈H₁₈N₂O₇ | 254.11252 | 8.314 | — |
| 59 | — | — | 255.16795 | 2.895 | 55.8, 70.0, 70.9, |
| | | | | | 71.9, 72.8, 73.8, |
| | | | | | 74.7, 78.7, 81.1, |
| | | | | | 83.9, 86.1, 86.9, |
| | | | | | 87.9, 88.9, 89.7, |
| | | | | | 90.9, 91.8, 100.8, |
| | | | | | 101.9, 102.8, |
| | | | | | 103.7, 105.3, |
| | | | | | 106.1, 106.7, |
| | | | | | 107.9, 108.8, |
| | | | | | 111.9, 112.5, |
| | | | | | 114.0, 115.9, |
| | | | | | 116.9, 118.0, |
| | | | | | 118.9, 120.1, |
| | | | | | 120.7, 125.9, |
| | | | | | 126.8, 128.0, |
| | | | | | 129.9, 130.8, |
| | | | | | 131.8, 132.8, |
| | | | | | 133.9, 134.9, |
| | | | | | 136.0, 140.0, |
| | | | | | 141.0, 141.9, |
| | | | | | 143.8, 145.1, |
| | | | | | 145.9, 147.0, |
| | | | | | 147.9, 148.8, |
| | | | | | 151.0, 151.8, |
| | | | | | 152.9, 159.9, |
| | | | | | 162.0, 163.9, |
| | | | | | 164.9, 165.9, |
| | | | | | 167.6, 168.9, |
| | | | | | 175.9, 176.9, |
| | | | | | 177.9, 188.7, |
| | | | | | 207.7, 209.8 |
| 60 | — | C₁₂H₂₄N₄S | 256.17125 | 7.054 | — |
| 61 | — | — | 257.19858 | 5.334 | 56.8, 68.9, 83.9, |
| | | | | | 102.0, 107.1, |
| | | | | | 109.5, 118.9, |
| | | | | | 122.9, 125.2, |
| | | | | | 128.1, 149.0, |
| | | | | | 153.3, 158.0, |
| | | | | | 160.8, 169.9, |
| | | | | | 176.1, 185.1, |
| | | | | | 194.1 |
| 62 | N-lauroylglycine | C₁₄H₂₇NO₃ | 257.19858 | 4.886 | 55.9, 56.9, 57.8, |
| | | | | | 81.3, 83.9, 85.1, |
| | | | | | 102.1, 123.3, |
| | | | | | 139.9, 143.7, |
| | | | | | 145.2, 176.0, |
| | | | | | 177.1, 181.0, |
| | | | | | 187.2, 250.7 |
| 63 | — | C₉H₁₈N₄O₅ | 262.12746 | 7.01 | — |
| 64 | — | — | 268.59456 | 2.059 | — |
| 65 | — | — | 269.18347 | 7.039 | — |
| 66 | — | C₈H₁₈N₂O₈ | 270.10737 | 7.978 | — |
| 67 | — | C₁₄H₁₆N₂O₂S | 276.09734 | 6.995 | 56.9, 81.1, 84.5, |
| | | | | | 101.0, 107.2, |
| | | | | | 121.0, 126.9, |
| | | | | | 135.2, 151.1, |
| | | | | | 155.8, 174.8, |
| | | | | | 176.0, 177.0, |
| | | | | | 178.7, 205.5, |
| | | | | | 235.2, 277.0 |
| 68 | — | — | 276.29762 | 7.056 | 56.9, 132.8, |
| | | | | | 148.9, 156.9, |
| | | | | | 158.0, 162.5, |
| | | | | | 164.3, 174.8, |
| | | | | | 179.2 |
| 69 | — | C₈H₂₄ClN₉ | 281.18376 | 7.012 | — |
| 70 | — | — | 283.19892 | 7.043 | 63.0, 72.9, 87.1, |
| | | | | | 89.0, 89.9, 97.1, |
| | | | | | 107.2, 116.9, |
| | | | | | 133.0, 136.0, |
| | | | | | 151.1, 153.8, |
| | | | | | 176.9, 182.2, |
| | | | | | 195.0, 239.0 |
| 71 | — | C₁₄H₁₆N₄O₃ | 288.11788 | 3.909 | 56.8, 94.9, 117.1, |
| | | | | | 119.4, 149.4, |
| | | | | | 154.9, 156.6, |
| | | | | | 171.0, 176.4, |
| | | | | | 181.0, 199.4, |
| | | | | | 289.1 |
| 72 | — | C₄H₇N₂O₁₁P | 289.97981 | 1.338 | 68.9, 84.8, 91.0, |
| | | | | | 115.1, 142.9, |
| | | | | | 160.9, 165.0, |
| | | | | | 178.2, 187.2, |
| | | | | | 216.5 |
| 73 | — | C₁₀H₁₇NO₇S | 295.07271 | 3.621 | — |
| 74 | — | C₁₈H₁₆O₄ | 296.10465 | 3.599 | 55.0, 56.9, 58.8, |
| | | | | | 64.9, 67.2, 70.8, |
| | | | | | 71.7, 75.0, 77.0, |
| | | | | | 78.9, 90.0, 91.0, |
| | | | | | 93.0, 94.9, 101.9, |
| | | | | | 103.0, 105.0, |
| | | | | | 107.1, 107.8, |
| | | | | | 111.0, 116.3, |
| | | | | | 121.0, 125.8, |
| | | | | | 131.2, 148.8, |
| | | | | | 150.9 |
| 75 | — | — | 297.17815 | 7.306 | 69.1, 71.2, 72.9, |
| | | | | | 87.4, 88.6, 102.9, |
| | | | | | 104.8, 113.1, |
| | | | | | 119.1, 131.0, |
| | | | | | 133.0, 157.7, |
| | | | | | 170.0, 173.5, |
| | | | | | 197.8 |
| 76 | — | C₁₇H₂₀N₂OS | 300.11791 | 5.213 | 54.6, 109.2, |
| | | | | | 146.9, 154.7, |
| | | | | | 171.0, 177.0 |
| 77 | — | C₁₅H₂₇NO₅ | 301.18834 | 4.974 | 56.8, 58.0, 62.0, |
| | | | | | 75.0, 81.0, 83.9, |
| | | | | | 84.8, 102.0, |
| | | | | | 102.9, 114.0, |
| | | | | | 123.1, 128.0, |
| | | | | | 162.6, 166.8, |
| | | | | | 188.7 |
| 78 | — | C₁₀H₁₈N₆O₅ | 302.13353 | 4.086 | 57.0, 66.8, 72.9, |
| | | | | | 77.0, 84.6, 96.0, |
| | | | | | 96.8, 98.8, 114.9, |
| | | | | | 123.0, 131.0, |
| | | | | | 146.9, 148.7, |
| | | | | | 156.3, 159.6, |
| | | | | | 175.0, 177.3, |
| | | | | | 181.1, 211.0, |
| | | | | | 222.9, 303.1 |
| 79 | — | C₁₀H₁₆O₇P₂ | 310.03754 | 3.61 | — |
| 80 | — | — | 322.09363 | 7.05 | — |
| 81 | — | C₁₆H₂₉NO₆ | 331.1989 | 6.775 | — |
| 82 | — | C₂₀H₁₄O₅ | 334.08355 | 6.765 | 56.6, 129.9, |
| | | | | | 148.9, 173.9, |
| | | | | | 187.9, 191.7, |
| | | | | | 215.0, 217.3, |
| | | | | | 247.0 |
| 83 | — | C₉H₁₂N₃O₉P | 337.03072 | 1.545 | 74.1, 100.8, |
| | | | | | 101.8, 117.2, |
| | | | | | 131.1, 133.8, |
| | | | | | 142.7, 145.8, |
| | | | | | 147.0, 147.9, |
| | | | | | 148.9, 155.8, |
| | | | | | 156.9, 160.9, |
| | | | | | 165.8, 169.8, |
| | | | | | 172.1, 174.3, |
| | | | | | 175.2, 176.2, |
| | | | | | 178.9, 181.0, |
| | | | | | 190.8, 201.1, |
| | | | | | 202.3, 280.3, |
| | | | | | 338.1 |
| 84 | — | C₁₈H₂₇NO₃S | 337.17098 | 1.665 | 55.8, 67.9, 69.7, |
| | | | | | 72.8, 83.9, 85.8, |
| | | | | | 86.9, 87.8, 91.0, |
| | | | | | 101.9, 102.7, |
| | | | | | 107.7, 112.0, |
| | | | | | 113.8, 114.9, |
| | | | | | 115.8, 118.1, |
| | | | | | 130.0, 130.9, |
| | | | | | 131.8, 132.9, |
| | | | | | 133.8, 134.8, |
| | | | | | 137.8, 140.1, |
| | | | | | 144.0, 145.8, |
| | | | | | 147.1, 148.0, |
| | | | | | 148.9, 149.9, |
| | | | | | 150.7, 151.8, |
| | | | | | 155.9, 156.6, |
| | | | | | 157.7, 158.8, |
| | | | | | 159.6, 161.9, |
| | | | | | 163.9, 164.9, |
| | | | | | 165.8, 167.8, |
| | | | | | 171.9, 172.7, |
| | | | | | 173.9, 174.8, |
| | | | | | 175.8, 176.7, |
| | | | | | 177.9, 179.0, |
| | | | | | 179.8, 183.6, |
| | | | | | 190.1, 191.9, |
| | | | | | 201.8, 216.0, |
| | | | | | 219.8, 234.8, |
| | | | | | 238.0, 245.7 |
| 85 | — | C₁₅H₁₈N₆O₅ | 362.13355 | 4.884 | — |
| 86 | — | — | 393.09001 | 3.625 | — |
| 87 | — | — | 415.07206 | 3.62 | — |
| 88 | — | — | 432.10054 | 6.762 | — |
| 89 | — | C₂₄H₃₈N₈ | 438.32205 | 1.942 | 56.8, 62.0, 83.9, |
| | | | | | 85.0, 102.0, |
| | | | | | 123.0, 146.1, |
| | | | | | 202.1 |
| 90 | — | — | 698.41818 | 7.045 | — |
| 91 | — | C₅H₁₀O₃ | 118.0623 | 5.382 | 59.0, 71.0, 73.8, |
| | | | | | 74.8, 99.9, 116.9 |
| 92 | — | C₁H₂S₁Br₁ | 124.9087 | 24.875 | 78.7, 81.9, 87.9, |
| | | | | | 94.1, 105.9, |
| | | | | | 123.9 |
| 93 | — | C₉H₁₇N₁O₅ | 219.1099 | 3.968 | 70.8, 71.9, 87.9, |
| | | | | | 98.8, 116.1, |
| | | | | | 127.8, 146.1, |
| | | | | | 218.1 |
| 94 | — | C₃H₃O₂S₂P₁Cl₂ | 235.8662 | 1.938 | 58.9, 70.9, 73.8, |
| | | | | | 87.0, 89.0, 92.8, |
| | | | | | 94.7, 101.1, |
| | | | | | 103.0, 108.3, |
| | | | | | 110.5, 112.8, |
| | | | | | 117.0, 131.0, |
| | | | | | 133.2, 136.7, |
| | | | | | 144.7, 148.9, |
| | | | | | 152.5, 154.7, |
| | | | | | 160.6, 162.8, |
| | | | | | 171.1, 174.7, |
| | | | | | 176.9, 191.1, |
| | | | | | 234. |
| 95 | — | C₁₃H₁₄N₂O₄ | 262.0947 | 3.758 | 56.9, 57.8, 73.8, |
| | | | | | 84.1, 87.0, 92.8, |
| | | | | | 98.2, 102.8, |
| | | | | | 110.9, 116.0, |
| | | | | | 117.9, 124.8, |
| | | | | | 126.9, 128.0, |
| | | | | | 130.1, 131.0, |
| | | | | | 132.0, 132.8, |
| | | | | | 140.1, 141.0, |
| | | | | | 144.0, 146.0, |
| | | | | | 157.1, 157.9, |
| | | | | | 158.7, 170.6, |
| | | | | | 175.0, 192.8, |
| | | | | | 218.8 |
| 96 | — | C₇H₁₀N₁₀O₂ | 266.0995 | 2.646 | 79.0, 94.8, 99.0, |
| | | | | | 118.9, 121.1, |
| | | | | | 126.2, 135.1, |
| | | | | | 146.9, 160.9, |
| | | | | | 162.8, 174.8, |
| | | | | | 178.8, 191.0, |
| | | | | | 197.2 |
| 97 | — | C₈H₂₁N₃O₄S₂ | 287.0973 | 3.966 | 88.1, 99.1, 117.2, |
| | | | | | 146.0, 151.9, |
| | | | | | 154.0 |
| 98 | — | C₁₂H₁₀N₆O₄ | 302.0761 | 2.635 | 58.9, 88.7, 101.0, |
| | | | | | 108.8, 114.8, |
| | | | | | 118.8, 136.7, |
| | | | | | 147.0, 152.9, |
| | | | | | 156.8, 164.9, |
| | | | | | 174.8, 186.9, |
| | | | | | 209.0 |
| 99 | — | C₁₄H₃₀O₅S₁ | 310.1809 | 24.987 | 79.7, 80.7, 94.8, |
| | | | | | 97.0, 98.0, 104.8, |
| | | | | | 114.8, 117.0, |
| | | | | | 117.8, 122.9, |
| | | | | | 128.7, 130.9, |
| | | | | | 150.8, 154.8, |
| | | | | | 163.1, 167.2, |
| | | | | | 172.1, 173.2, |
| | | | | | 174.6, 182.9, |
| | | | | | 205.2, 209.0, |
| | | | | | 222.9, 309.1, |
| | | | | | 309.8 |
| 100 | — | C₆H₁₆N₄O₁S₁ | 312.105 | 2.638 | 58.9, 88.9, 91.1, |
| | | | | | 99.0, 119.0, |
| | | | | | 131.1, 134.8, |
| | | | | | 137.1, 146.9, |
| | | | | | 152.9, 163.1, |
| | | | | | 174.9, 180.7, |
| | | | | | 241.2, 244.8 |
| 101 | — | C₇H₅O₁₀S₂P₃ | 405.8534 | 24.829 | — |
| 102 | — | C₅H₈N₆O₁₄S₁ | 407.9821 | 20.335 | — |

TABLE 2B

| NO. | Structure | Name | Formula |
|---|---|---|---|
| 2 | 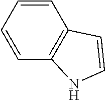 | Indole | C₈H₇N |
| 4 | 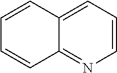 | Leucoline | C₉H₇N |
| 5 | 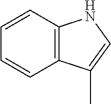 | Skatole | C₉H₉N |
| 6 | 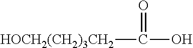 | 6-Hydroxycaproic acid | C₆H₁₂O₃ |
| 7 | 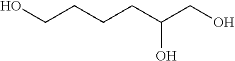 | 1,2,6-Hexanetriol | C₆H₁₄O₃ |
| 9 | 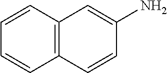 [SMILES: Nc1ccc2ccccc2c1] | Naphthalen-2-amine | C₁₀H₉N |
| 10 | 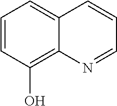 [SMILES: Oc1cccc2cccnc12] | 8-Hydroxyquinoline | C₉H₇NO |
| 13 | 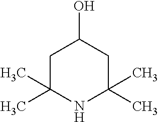 | 2,2,6,6-Tetramethyl-4-piperidinol | C₉H₁₉NO |
| 14 | 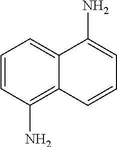 [SMILES: Nc1cccc2c(N)cccc12] | 1,5-Naphthalenediamine | C₁₀H₁₀N₂ |
| 15 | 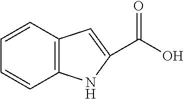 [SMILES: O=C(O)c1cc2ccccc2[nH]1] | 1H-Indole-2-carboxylic acid | C₉H₇NO₂ |
| 19 | 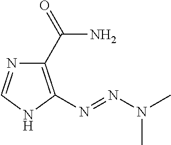 | (E)-Dacarbazine | C₆H₁₀N₆O |
| 21 | 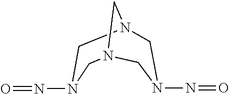 [SMILES: O=NN1CN2CN(C1)CN(N=O)C2] | Dinitrosopentamethylenetetramine | C₅H₁₀N₆O₂ |
| 23 | 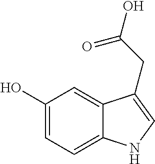 [SMILES: O=C(O)Cc1c[nH]c2ccc(O)cc12] | 5-Hydroxyindole-3-Acetic Acid | C₁₀H₉NO₃ |
| 25 | 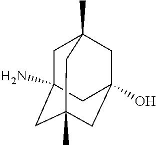 | 3-Amino-5,7-dimethyl-1- adamantanol | C₁₂H₂₁NO |
| 27 | 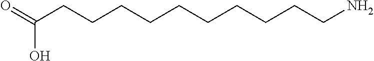 [SMILES: NCCCCCCCCCCC(=O)O] | 11-Aminoundecanoic acid | C₁₁H₂₃NO₂ |
| 31 | 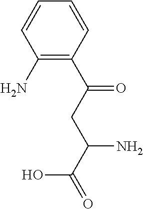 [SMILES: Nc1ccccc1C(=O)CC(N)C(=O)O] | Kynurenine | C₁₀H₁₂N₂O₃ |
| 38 | 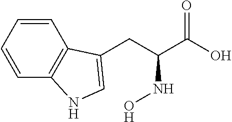 [SMILES: O=C(O)[C@H](Cc1c[nH]c2ccccc12)NO] | N-Hydroxy-L-tryptophan | C₁₁H₁₂N₂O₃ |
| 39 | 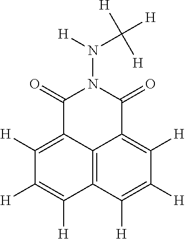 [SMILES: CNN1C(=O)c2cccc3cccc(c23)C1=O] | 2-(Methylamino)-1H- benzo[de]isoquinoline-1,3(2H)- dione | C₁₃H₁₀N₂O₂ |
| 56 | 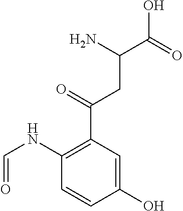 [SMILES: NC(CC(=O)c1cc(O)ccc1NC=O)C(=O)O] | 5-Hydroxy-N-formylkynurenine | C₁₁H₁₂N₂O₅ |
| 62 | 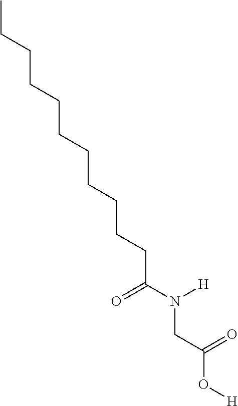 [SMILES: CCCCCCCCCCCC(=O)NCC(=O)O] | N-Lauroylglycine | C₁₄H₂₇NO₃ |

### EXAMPLES

### Example 1: Identification of Pregnancy and Non-Pregnancy Biomarkers

### Sample Collection.

Embryo-conditioned Vitrolife media was collected on day 5 of culture following fertilization and frozen for storage at −20° C.

### Sample Preparation

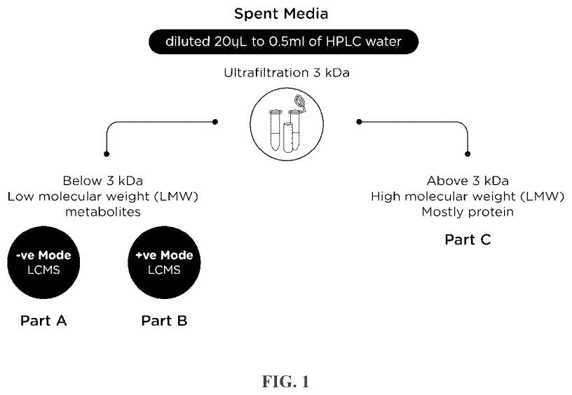

Frozen embryo-conditioned media was thawed at 4° C. overnight. A total of 20 μL of embryo-conditioned media was then added to 480 μL of HPLC grade water in Amicon® Ultra 0.5-mL 3-kDa centrifugal filters. Samples were then spun by centrifuge at 4,000×g for 10 min to separate proteins from metabolites. A schematic of this process is shown in FIG. 1. Following centrifugation, the metabolite fraction was present in the solution that passed through the Amicon® filter, while protein was present in the filter membrane. Both the metabolite fraction and protein extract were then separately collected and frozen at −80° C. for storage and future analysis.

### Metabolomics Analysis by UPLC-Orbitrap Fusion Mass Spectroscopy

Untargeted metabolomics analysis was conducted using a Vanquish UPLC-Orbitrap Fusion Tribrid Mass Spectrometer (UPLC-OT-FTMS). Samples were passed through a 1.7-μm ACQUITY UPLC BEH C18 reverse phase column (Waters, 30 Å, 1.7 μm, 2.1 mm×100 mm) to an Orbitrap Fusion Tribrid Mass Spectrometer (Thermo Scientific) operated under both positive and negative modes via a heated ESI source. For the UPLC step, the following gradient was used: hold at 5% Eluent B (acetonitrile with 0.1% (v/v) formic acid) and 95% Eluent A (Milli-Q water with 0.1% (v/v) formic acid) for 2 min; ramp to 65% B, 35% A for 16 min; ramp to 100% B for 7 min and hold for 8 min. An 8 min column re-equilibration with the starting ratio of eluents was carried out between sample analyses. Throughout the process a flow rate of 0.2 mL/min and an injection volume of 20 μL were used.

For negative mode analysis, the ESI setting was 3,000 volts, 30 AU sheath gas, 10 AU Aux gas, 0 AU sweep gas, a 325° C. ion transfer tube temperature and a 200° C. vaporizer temperature. The Orbitrap full scan was run at 500,000 resolutions with a scan range 100-1000 m/z and radio frequency (RF) Lens at 40%. For MS², the isolation window was set at 0.7 m/z while performing both CID and HCD using an ion trap mass spectrometer as the detector. The automatic gain control (AGC) was set at 10,000 with an intensity threshold at 5,000.

Compound discoverer software 2.1 (Thermo Fisher) was used to identify the metabolomics compounds and to perform the multivariate analyses (Volcano plot, Principal Components Analysis (PCA), Partial Least Squares Discriminant Analysis (PLS-DA) and trend analysis). In brief, all the raw spectra were uploaded to the software, and retention times were aligned for all spectra. Mass features in each individual spectrum were then detected. The molecular formula for each peak was calculated with the following parameters: C₄₋₅₀H₄₋₂₀₀O₀₋₂₀N₀₋₈S₀₋₂P₀₋₂. Molecular formulas that are unlikely to occur in nature, or are not chemically possible, were then removed. For the natural abundance of ¹³C relative to ¹²C, a value of 1.11% was used. For any compound containing phosphorous, a requirement that at least three oxygen atoms per each phosphorus atom must exist was set. Based on this analysis, compound formulas were assigned within 1 ppm error.

The normalized integrated area of mass spectra for each compound was used to perform volcano plots and trend analysis. De novo compound structure elucidation was identified based on MS/MS and confirmed using in silico fragmentation prediction software (e.g. Mass Frontier, MetFrag, and ACD/MS fragmenter).

### Results of Biomarker Identification.

A total of 47 embryo-conditioned Vitrolife media samples were analyzed by liquid chromatography-MS (LC-MS). The media samples were separated into 3 groups based on known clinical outcomes: 1) not pregnant (NP; 17 samples), 2) pregnant (P; 20 samples), and 3) blank control media (C; 10 samples). Over 8,000 single chemical compounds were identified in conditioned media groups.

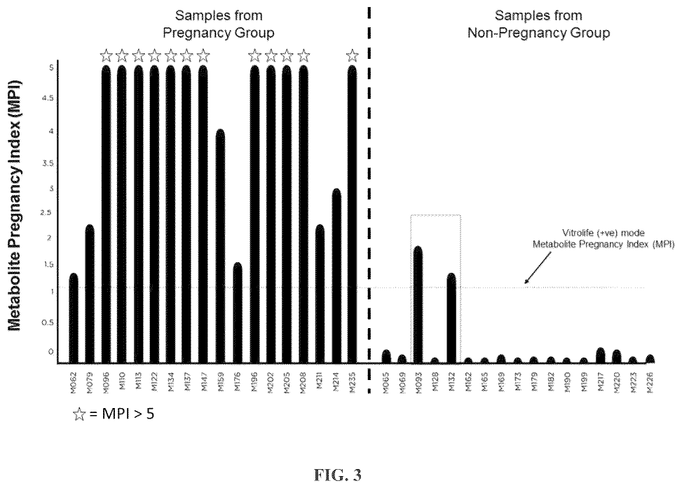

To identify metabolites that were differentially present in P vs. NP samples, the relative abundance of metabolites in P vs. NP samples was determined (in terms of fold change) and analyzed with volcano plots. This analysis was performed for results obtained with both positive mode and negative mode MS methods. As shown in FIG. 3 and TABLE 3, 154 potential biomarkers of pregnancy and non-pregnancy were identified. Cutoff values for the identification of a metabolite as a pregnancy or non-pregnancy biomarker are also shown in TABLE 3. Using the tandem MS methods described in the preceding section, details on the chemical name, molecular weight, chemical formula, retention time, fragmentation pattern, and/or chemical structure of pregnant and non-pregnant biomarkers were determined and are shown above in TABLES 1A, 1B, 2A, and 2B.

TABLE 3

| | Total | Number of | | | | Cut-off- | Cut-off- |
|---|---|---|---|---|---|---|---|
| | number of | Biomarkers | | Biomarker | | log2 | log10 |
| Media | biomarkers | per mode | | per class | | FC | p-value |
| Vitrolife | 154 | Positive | 126 | P | 36 | 1.00 | 2.00 |
| | | | | NP | 90 | −1.00 | 2.00 |
| | | Negative | 28 | P | 16 | 2.00 | 2.00 |
| | | | | NP | 12 | −2.00 | 2.00 |

FC = Fold change

### Prediction of Implantation Probability.

Based on the relative abundances of pregnancy and non-pregnancy biomarkers in a conditioned-media sample, the probability of implantation success of the corresponding embryo can be assessed. To facilitate an assessment of embryo implantation success probability, a Metabolite Pregnancy Index (MPI) was developed. MPI is calculated by dividing the total integrated area of mass spectra peaks corresponding to selected pregnancy biomarkers by the total integrated area of mass spectra peaks corresponding to selected non-pregnancy biomarkers. MPI calculations can be based on the presence of anywhere from 1 to infinite numbers of pregnant/non-pregnant biomarkers. In the present example, a MPI was calculated based on the 154 identified biomarkers, and a pregnancy/non-pregnancy value of MPI=1.0 was set, with a MPI>1.0 indicating an embryo with a high probability of implantation success. Based on these parameters, results showed that 100% (20/20) of embryos that implanted (P samples) had a MPI>1.0. Of these 20 embryos, 8 had an MPI between 1-10 and 12 had an MPI>200. Of the embryos that did not implant (NP samples), 88% (15/17) had an MPI<1.0, while 12% (2/17) had an MPI from 1-2. The average MPI of NP samples was 0.068. These results are summarized in FIG. 3. From these results samples can be classified into three groups: 1) Those with a very high probability of implantation success (MPI>10), 2) those with a high probability of implantation success: (MPI from 1-10, and 3) those with a low probability of implantation success (MPI<1).

### Embodiments

The following non-limiting embodiments provide illustrative examples of methods of the disclosure but do not limit the scope of methods of the disclosure.

Embodiment 1. A method comprising: a) measuring an amount of a metabolite in a sample of culture media in which an embryo has been cultured in vitro, wherein the metabolite is selected from the group consisting of caprolactam, N-cyclohexylformamide, threo-3-hydroxy-2-methylbutyric acid, sulfoacetic acid, (2,7-dimethyloctahydro-1H-cyclopenta[c]pyridin-4-yl)methanol, trans-3-indoleacrylic acid, pantothenic acid, diaveridine, (5-benzyl-3,6-dioxo-2-piperazinyl)acetic acid, dasytrichone, indole, leucoline, skatole, 6-hydroxycaproic acid, 1,2,6-hexanetriol, naphthalen-2-amine, 8-hydroxyquinoline, 2,2,6,6-tetramethyl-4-piperidinol, 1,5-naphthalenediamine, 1H-indole-2-carboxylic acid, (E)-dacarbazine, dinitrosopentamethylenetetramine, 5-hydroxyindole-3-acetic acid, 3-amino-5,7-dimethyl-1-adamantanol, 11-aminoundecanoic acid, kynurenine, N-hydroxy-L-tryptophan, 2-(methylamino)-1H-benzo[de]isoquinoline-1,3(2H)-dione, 5-hydroxy-N-formylkynurenine, and N-lauroylglycine; and b) assessing a likelihood of success of implantation of the embryo into a uterus upon introduction of the embryo into the uterus based at least partially on the amount of the metabolite present in the sample of culture media.

Embodiment 2. The method of embodiment 1, wherein the metabolite is carpolactam.

Embodiment 3. The method of embodiment 1, wherein the metabolite is N-cyclohexylformamide.

Embodiment 4. The method of embodiment 1, wherein the metabolite is threo-3-hydroxy-2-methylbutyric acid.

Embodiment 5. The method of embodiment 1, wherein the metabolite is sulfoacetic acid.

Embodiment 6. The method of embodiment 1, wherein the metabolite is (2,7-dimethyloctahydro-1H-cyclopenta[c]pyridin-4-yl)methanol.

Embodiment 7. The method of embodiment 1, wherein the metabolite is trans-3-indoleacrylic acid.

Embodiment 8. The method of embodiment 1, wherein the metabolite is pantothenic acid.

Embodiment 9. The method of embodiment 1, wherein the metabolite is diaveridine.

Embodiment 10. The method of embodiment 1, wherein the metabolite is (5-benzyl-3,6-dioxo-2-piperazinyl)acetic acid.

Embodiment 11. The method of embodiment 1, wherein the metabolite is dasytrichone,

Embodiment 12. The method of embodiment 1, wherein the metabolite is indole.

Embodiment 13. The method of embodiment 1, wherein the metabolite is leucoline.

Embodiment 14. The method of embodiment 1, wherein the metabolite is skatole.

Embodiment 15. The method of embodiment 1, wherein the metabolite is 6-hydroxycaproic acid.

Embodiment 16. The method of embodiment 1, wherein the metabolite is 1,2,6-hexanetriol.

Embodiment 17. The method of embodiment 1, wherein the metabolite is naphthalen-2-amine.

Embodiment 18. The method of embodiment 1, wherein the metabolite is 8-hydroxyquinoline.

Embodiment 19. The method of embodiment 1, wherein the metabolite is 2,2,6,6-tetramethyl-4-piperidinol.

Embodiment 20. The method of embodiment 1, wherein the metabolite is 1,5-naphthalenediamine.

Embodiment 21. The method of embodiment 1, wherein the metabolite is 1H-indole-2-carboxylic acid.

Embodiment 22. The method of embodiment 1, wherein the metabolite is (E)-dacarbazine.

Embodiment 23. The method of embodiment 1, wherein the metabolite is dinitrosopentamethylenetetramine.

Embodiment 24. The method of embodiment 1, wherein the metabolite is 5-hydroxyindole-3-acetic acid.

Embodiment 25. The method of embodiment 1, wherein the metabolite is 3-amino-5,7-dimethyl-1-adamantanol.

Embodiment 26. The method of embodiment 1, wherein the metabolite is 11-aminoundecanoic acid.

Embodiment 27. The method of embodiment 1, wherein the metabolite is kynurenine.

Embodiment 28. The method of embodiment 1, wherein the metabolite is N-hydroxy-L-tryptophan.

Embodiment 29. The method of embodiment 1, wherein the metabolite is 2-(methylamino)-1H-benzo[de]isoquinoline-1,3(2H)-dione.

Embodiment 30. The method of embodiment 1, wherein the metabolite is 5-hydroxy-N-formylkynurenine.

Embodiment 31. The method of embodiment 1, wherein the metabolite is N-lauroylglycine.

Embodiment 32. A method comprising measuring an amount of each of a plurality of metabolites in a mixture, wherein each metabolite is one of the following compounds: (2,7-dimethyloctahydro-1H-cyclopenta[c]pyridin-4-yl)methanol, trans-3-indoleacrylic acid, pantothenic acid, diaveridine, (5-benzyl-3,6-dioxo-2-piperazinyl)acetic acid, dasytrichone, indole, skatole, 8-hydroxyquinoline, (E)-dacarbazine, kynurenine, or N-hydroxy-L-tryptophan.

Embodiment 33. The method of embodiment 32, wherein the mixture is a sample of culture media in which an embryo has been cultured in vitro.

Embodiment 34. The method of embodiment 32 or 33, further comprising assessing a likelihood of success of implantation of the embryo based on the amount of at least one metabolite in the mixture.

Embodiment 35. The method of any one of embodiments 32-34, further comprising measuring an amount of caprolactam in the mixture.

Embodiment 36. The method of any one of embodiments 32-35, further comprising measuring an amount of N-cyclohexylformamide in the mixture.

Embodiment 37. The method of any one of embodiments 32-36, further comprising measuring an amount of threo-3-hydroxy-2-methylbutyric acid in the mixture.

Embodiment 38. The method of any one of embodiments 32-37, further comprising measuring an amount of sulfoacetic acid in the mixture.

Embodiment 39. The method of any one of embodiments 32-38, further comprising measuring an amount of phenylalanine in the mixture.

Embodiment 40. The method of any one of embodiments 32-39, further comprising measuring an amount of DL-tryptophan in the mixture.

Embodiment 41. The method of any one of embodiments 32-40, further comprising measuring an amount of leucoline in the mixture.

Embodiment 42. The method of any one of embodiments 32-41, further comprising measuring an amount of 6-hydroxycaproic acid in the mixture.

Embodiment 43. The method of any one of embodiments 32-42, further comprising measuring an amount of 1,2,6-hexanetriol in the mixture.

Embodiment 44. The method of any one of embodiments 32-43, further comprising measuring an amount of naphthalen-2-amine in the mixture.

Embodiment 45. The method of any one of embodiments 32-44, further comprising measuring an amount of 8-hydroxyquinoline in the mixture.

Embodiment 46. The method of any one of embodiments 32-45, further comprising measuring an amount of 2,2,6,6-tetramethyl-4-piperidinol in the mixture.

Embodiment 47. The method of any one of embodiments 32-46, further comprising measuring an amount of 1,5-naphthalenediamine in the mixture.

Embodiment 48. The method of any one of embodiments 32-47, further comprising measuring an amount of 1H-indole-2-carboxylic acid in the mixture.

Embodiment 49. The method of any one of embodiments 32-48, further comprising measuring an amount of dinitrosopentamethylenetetramine in the mixture.

Embodiment 50. The method of any one of embodiments 32-49, further comprising measuring an amount of hydroxyindole-3-acetic acid in the mixture.

Embodiment 51. The method of any one of embodiments 32-50, further comprising measuring an amount of 3-amino-5,7-dimethyl-1-adamantanol in the mixture.

Embodiment 52. The method of any one of embodiments 32-51, further comprising measuring an amount of 11-aminoundecanoic acid in the mixture.

Embodiment 53. The method of any one of embodiments 32-52, further comprising measuring an amount of kynurenine in the mixture.

Embodiment 54. The method of any one of embodiments 32-53, further comprising measuring an amount of 2-(methylamino)-1H-benzo[de]isoquinoline-1,3(2H)-dione in the mixture.

Embodiment 55. The method of any one of embodiments 32-54, further comprising measuring an amount of 5-hydroxy-N-formylkynurenine in the mixture.

Embodiment 56. The method of any one of embodiments 32-55, further comprising measuring an amount of N-lauroylglycine in the mixture.

Embodiment 57. A method comprising: a) performing an assay to obtain a first set of mass spectra corresponding to a first metabolite present in a sample of culture media in which an embryo has been cultured in vitro (ms₁); b) using the first set of mass spectra to determine an amount of the first metabolite in the sample of culture media, wherein the amount of the first metabolite is associated with a low probability of embryo implantation success; c) performing an assay to obtain a second set of mass spectra corresponding to a second metabolite present in the sample of culture media in which the embryo has been cultured in vitro (ms₂); d) using the second set of mass spectra to determine an amount of the second metabolite, wherein the amount of the second metabolite is associated with a high probability of embryo implantation success; and comparing the amount of the first metabolite with the amount of the second metabolite.

Embodiment 58. The method of embodiment 57, wherein the amount of the first metabolite is compared to the amount of the second metabolite by solving the equation:

$$MPI = \frac{\int {ms}_{2}}{\int {ms}_{1}} .$$

Embodiment 59. The method of embodiment 57 or 58, further comprising determining an amount of each of 5 additional metabolites in the sample of culture media, wherein the 5 additional metabolites are associated with a low probability of embryo implantation success.

Embodiment 60. The method of any one of embodiments 57-59, further comprising determining an amount of each of 5 additional metabolites in the sample of culture media, wherein the 5 additional metabolites are associated with a high probability of embryo implantation success.

Embodiment 61. The method of any one of embodiments 57, 58, and 60, further comprising determining an amount of each of 19 additional metabolites in the sample of culture media, wherein the 19 additional metabolites are associated with a low probability of embryo implantation success.

Embodiment 62. The method of any one of embodiments 57-59 and 61, further comprising determining an amount of each of 12 additional metabolites in the sample of culture media, wherein the 12 additional metabolites are associated with a high probability of embryo implantation success.

Embodiment 63. The method of any one of embodiments 57, 58, 60, and 62, further comprising determining an amount of each of 19-93 additional metabolites in the sample of culture media, wherein the 19-93 additional metabolites are associated with a low probability of embryo implantation success.

Embodiment 64. The method of any one of embodiments 57-59, 61, and 63, further comprising determining an amount of each of 11-50 additional metabolites in the sample of culture media, wherein the 11-50 additional metabolites are associated with a high probability of embryo implantation success.

Embodiment 65. The method of any one of embodiments 57, 58, 60, 62, and 64, further comprising determining an amount of each of 19-101 additional metabolites in the sample of culture media, wherein the 19-101 additional metabolites are associated with a low probability of embryo implantation success.

Embodiment 66. The method of any one of embodiments 57-59, 61, 63, and 65, further comprising determining an amount of each of 11-51 additional metabolites in the sample of culture media, wherein the 11-51 additional metabolites are associated with a high probability of embryo implantation success.

Embodiment 67. A method comprising: a) measuring an amount of each of 4 metabolites in a sample of culture media in which an embryo has been cultured in vitro; and b) assessing a likelihood of success of implantation of the embryo into a uterus upon introduction of the embryo into the uterus based on the amount of the 4 metabolites in the sample of culture media, wherein: i) the amount of at least one of the metabolites in the sample of culture media is associated with a low probability of embryo implantation success; and ii) the amount of at least one of the metabolites in the sample of culture media is associated with a high probability of implantation success.

Embodiment 68. The method of embodiment 67, wherein one of the metabolites is caprolactam.

Embodiment 69. The method of embodiment 67 or 68, wherein one of the metabolites is N-cyclohexylformamide.

Embodiment 70. The method of any one of embodiments 67-69, wherein one of the metabolites is threo-3-hydroxy-2-methylbutyric acid.

Embodiment 71. The method of any one of embodiments 67-70, wherein one of the metabolites is sulfoacetic acid.

Embodiment 72. The method of any one of embodiments 67-71, wherein one of the metabolites is (2,7-dimethyloctahydro-1H-cyclopenta[c]pyridin-4-yl)methanol.

Embodiment 73. The method of any one of embodiments 67-72, wherein one of the metabolites is trans-3-indoleacrylic acid.

Embodiment 74. The method of any one of embodiments 67-73, wherein one of the metabolites is pantothenic acid.

Embodiment 75. The method of any one of embodiments 67-74, wherein one of the metabolites is diaveridine.

Embodiment 76. The method of any one of embodiments 67-75, wherein one of the metabolites is (5-benzyl-3,6-dioxo-2-piperazinyl)acetic acid.

Embodiment 77. The method of any one of embodiments 67-76, wherein one of the metabolites is dasytrichone.

Embodiment 78. The method of any one of embodiments 67-77, wherein one of the metabolites is indole.

Embodiment 79. The method of any one of embodiments 67-78, wherein one of the metabolites is leucoline.

Embodiment 80. The method of any one of embodiments 67-79, wherein one of the metabolites is skatole.

Embodiment 81. The method of any one of embodiments 67-80, wherein one of the metabolites is 6-hydroxycaproic acid.

Embodiment 82. The method of any one of embodiments 67-81, wherein one of the metabolites is 1,2,6-hexanetriol.

Embodiment 83. The method of any one of embodiments 67-82, wherein one of the metabolites is naphthalen-2-amine.

Embodiment 84. The method of any one of embodiments 67-83, wherein one of the metabolites is 8-hydroxyquinoline.

Embodiment 85. The method of any one of embodiments 67-84, wherein one of the metabolites is 2,2,6,6-tetramethyl-4-piperidinol.

Embodiment 86. The method of any one of embodiments 67-85, wherein one of the metabolites is 1,5-naphthalenediamine.

Embodiment 87. The method of any one of embodiments 67-86, wherein one of the metabolites is 1H-indole-2-carboxylic acid.

Embodiment 88. The method of any one of embodiments 67-87, wherein one of the metabolites is (E)-dacarbazine.

Embodiment 89. The method of any one of embodiments 67-88, wherein one of the metabolites is dinitrosopentamethylenetetramine.

Embodiment 90. The method of any one of embodiments 67-89, wherein one of the metabolites is 5-hydroxyindole-3-acetic acid.

Embodiment 91. The method of any one of embodiments 67-90, wherein one of the metabolites is 3-amino-5,7-dimethyl-1-adamantanol.

Embodiment 92. The method of any one of embodiments 67-91, wherein one of the metabolites is 11-aminoundecanoic acid.

Embodiment 93. The method of any one of embodiments 67-92, wherein one of the metabolites is kynurenine.

Embodiment 94. The method of any one of embodiments 67-93, wherein one of the metabolites is N-hydroxy-L-tryptophan.

Embodiment 95. The method of any one of embodiments 67-94, wherein one of the metabolites is 2-(methylamino)-1H-benzo[de]isoquinoline-1,3(2H)-dione.

Embodiment 96. The method of any one of embodiments 67-95, wherein one of the metabolites is 5-hydroxy-N-formylkynurenine.

Embodiment 97. The method of any one of embodiments 67-96, wherein one of the metabolites is N-lauroylglycine.

Embodiment 98. The method of any one of embodiments 67-97, wherein one of the metabolites is DL-tryptophan.

Embodiment 99. The method of any one of embodiments 67-98, wherein one of the metabolites is phenylalanine.

Embodiment 100. A method comprising: a) requesting an assay on a sample of culture media in which an embryo has been cultured in vitro, b) receiving a communication, wherein the communication assesses a likelihood of success of implantation of the embryo into a uterus upon introduction of the embryo into the uterus based on an amount of one or more metabolites measured in the sample of culture media; and c) transferring the embryo into a subject's uterus, wherein: i) the communication states that the likelihood of success of implantation of the embryo into the uterus is high; and ii) one of the metabolites measured in the sample of culture media is caprolactam, N-cyclohexylformamide, threo-3-hydroxy-2-methylbutyric acid, sulfoacetic acid, (2,7-dimethyloctahydro-1H-cyclopenta[c]pyridin-4-yl)methanol, trans-3-indoleacrylic acid, pantothenic acid, diaveridine, (5-benzyl-3,6-dioxo-2-piperazinyl)acetic acid, dasytrichone, indole, leucoline, skatole, 6-hydroxycaproic acid, 1,2,6-hexanetriol, naphthalen-2-amine, 8-hydroxyquinoline, 2,2,6,6-tetramethyl-4-piperidinol, 1,5-naphthalenediamine, 1H-indole-2-carboxylic acid, (E)-dacarbazine, dinitrosopentamethylenetetramine, 5-hydroxyindole-3-acetic acid, 3-amino-5,7-dimethyl-1-adamantanol, 11-aminoundecanoic acid, kynurenine, N-hydroxy-L-tryptophan, 2-(methylamino)-1H-benzo[de]isoquinoline-1,3(2H)-dione, 5-hydroxy-N-formylkynurenine, or N-lauroylglycine.

Embodiment 101. The method of embodiment 100, wherein the likelihood of success of implantation of the embryo is based on the amount of caprolactam measured in the sample of culture media.

Embodiment 102. The method of embodiment 100, wherein the likelihood of success of implantation of the embryo is based on the amount of N-cyclohexylformamide measured in the sample of culture media.

Embodiment 103. The method of embodiment 100, wherein the likelihood of success of implantation of the embryo is based on the amount of threo-3-hydroxy-2-methylbutyric acid measured in the sample of culture media.

Embodiment 104. The method of embodiment 100, wherein the likelihood of success of implantation of the embryo is based on the amount of sulfoacetic acid measured in the sample of culture media

Embodiment 105. The method of embodiment 100, wherein the likelihood of success of implantation of the embryo is based on the amount of (2,7-dimethyloctahydro-1H-cyclopenta[c]pyridin-4-yl)methanol measured in the sample of culture media.

Embodiment 106. The method of embodiment 100, wherein the likelihood of success of implantation of the embryo is based on the amount of trans-3-indoleacrylic acid measured in the sample of culture media.

Embodiment 107. The method of embodiment 100, wherein the likelihood of success of implantation of the embryo is based on the amount of pantothenic acid measured in the sample of culture media.

Embodiment 108. The method of embodiment 100, wherein the likelihood of success of implantation of the embryo is based on the amount of diaveridine measured in the sample of culture media.

Embodiment 109. The method of embodiment 100, wherein the likelihood of success of implantation of the embryo is based on the amount of (5-benzyl-3,6-dioxo-2-piperazinyl)acetic acid measured in the sample of culture media.

Embodiment 110. The method of embodiment 100, wherein the likelihood of success of implantation of the embryo is based on the amount of dasytrichone measured in the sample of culture media.

Embodiment 111. The method of embodiment 100, wherein the likelihood of success of implantation of the embryo is based on the amount of indole measured in the sample of culture media.

Embodiment 112. The method of embodiment 100, wherein the likelihood of success of implantation of the embryo is based on the amount of leucoline measured in the sample of culture media.

Embodiment 113. The method of embodiment 100, wherein the likelihood of success of implantation of the embryo is based on the amount of skatole measured in the sample of culture media.

Embodiment 114. The method of embodiment 100, wherein the likelihood of success of implantation of the embryo is based on the amount of 6-hydroxycaproic acid measured in the sample of culture media.

Embodiment 115. The method of embodiment 100, wherein the likelihood of success of implantation of the embryo is based on the amount of 1,2,6-hexanetriol measured in the sample of culture media.

Embodiment 116. The method of embodiment 100, wherein the likelihood of success of implantation of the embryo is based on the amount of naphthalen-2-amine measured in the sample of culture media.

Embodiment 117. The method of embodiment 100, wherein the likelihood of success of implantation of the embryo is based on the amount of 8-hydroxyquinoline measured in the sample of culture media.

Embodiment 118. The method of embodiment 100, wherein the likelihood of success of implantation of the embryo is based on the amount of 2,2,6,6-tetramethyl-4-piperidinol measured in the sample of culture media.

Embodiment 119. The method of embodiment 100, wherein the likelihood of success of implantation of the embryo is based on the amount of 1,5-naphthalenediamine measured in the sample of culture media.

Embodiment 120. The method of embodiment 100, wherein the likelihood of success of implantation of the embryo is based on the amount of 1H-indole-2-carboxylic acid measured in the sample of culture media.

Embodiment 121. The method of embodiment 100, wherein the likelihood of success of implantation of the embryo is based on the amount of (E)-dacarbazine measured in the sample of culture media.

Embodiment 122. The method of embodiment 100, wherein the likelihood of success of implantation of the embryo is based on the amount of dinitrosopentamethylenetetramine measured in the sample of culture media.

Embodiment 123. The method of embodiment 100, wherein the likelihood of success of implantation of the embryo is based on the amount of 5-hydroxyindole-3-acetic acid measured in the sample of culture media.

Embodiment 124. The method of embodiment 100, wherein the likelihood of success of implantation of the embryo is based on the amount of 3-amino-5,7-dimethyl-1-adamantanol measured in the sample of culture media.

Embodiment 125. The method of embodiment 100, wherein the likelihood of success of implantation of the embryo is based on the amount of 11-aminoundecanoic acid measured in the sample of culture media.

Embodiment 126. The method of embodiment 100, wherein the likelihood of success of implantation of the embryo is based on the amount of kynurenine measured in the sample of culture media.

Embodiment 127. The method of embodiment 100, wherein the likelihood of success of implantation of the embryo is based on the amount of N-hydroxy-L-tryptophan measured in the sample of culture media.

Embodiment 128. The method of embodiment 100, wherein the likelihood of success of implantation of the embryo is based on the amount of 2-(methylamino)-1H-benzo[de]isoquinoline-1,3(2H)-dione measured in the sample of culture media.

Embodiment 129. The method of embodiment 100, wherein the likelihood of success of implantation of the embryo is based on the amount of 5-hydroxy-N-formylkynurenine measured in the sample of culture media.

Embodiment 130. The method of embodiment 100, wherein the likelihood of success of implantation of the embryo is based on the amount of N-lauroylglycine measured in the sample of culture media.

Embodiment 131. A kit comprising an aliquot of each of 3 of the following compounds: caprolactam, N-cyclohexylformamide, threo-3-hydroxy-2-methylbutyric acid, sulfoacetic acid, (2,7-dimethyloctahydro-1H-cyclopenta[c]pyridin-4-yl)methanol, trans-3-indoleacrylic acid, pantothenic acid, diaveridine, (5-benzyl-3,6-dioxo-2-piperazinyl)acetic acid, dasytrichone, indole, leucoline, skatole, 6-hydroxycaproic acid, 1,2,6-hexanetriol, naphthalen-2-amine, 8-hydroxyquinoline, 2,2,6,6-tetramethyl-4-piperidinol, 1,5-naphthalenediamine, 1H-indole-2-carboxylic acid, (E)-dacarbazine, dinitrosopentamethylenetetramine, 5-hydroxyindole-3-acetic acid, 3-amino-5,7-dimethyl-1-adamantanol, 11-aminoundecanoic acid, kynurenine, N-hydroxy-L-tryptophan, 2-(methylamino)-1H-benzo[de]isoquinoline-1,3(2H)-dione, 5-hydroxy-N-formylkynurenine, DL-tryptophan, phenylalanine, and N-lauroylglycine.

What is claimed is:

1. A method comprising:
a) obtaining a sample of culture media in which one or more embryos has been cultured in vitro;
b) measuring from the sample an amount of one or more metabolites in the sample, wherein at least one of the metabolites is selected from the group consisting of caprolactam, N-cyclohexylformamide, threo-3-hydroxy-2-methylbutyric acid, sulfoacetic acid, (2,7-dimethyloctahydro-1H-cyclopenta[c]pyridin-4-yl)methanol, trans-3-indoleacrylic acid, pantothenic acid, diaveridine, (5-benzyl-3,6-dioxo-2-piperazinyl)acetic acid, dasytrichone, indole, leucoline, skatole, 6-hydroxycaproic acid, 1,2,6-hexanetriol, naphthalen-2-amine, 8-hydroxyquinoline, 2,2,6,6-tetramethyl-4-piperidinol, 1,5-naphthalenediamine, 1H-indole-2-carboxylic acid, (E)-dacarbazine, dinitrosopentamethylenetetramine, 5-hydroxyindole-3-acetic acid, 3-amino-5,7-dimethyl-1-adamantanol, 11-aminoundecanoic acid, kynurenine, N-hydroxy-L-tryptophan, 2-(methylamino)-1H-benzo[de]isoquinoline-1,3(2H)-dione, 5-hydroxy-N formylkynurenine, and N-lauroylglycine; and
c) comparing the amount of the one or more metabolites in the sample to a reference of known clinical outcomes; and
d) transferring one or more embryos with a high probability of implantation success based on step c) into a subject's uterus.

2. The method of claim 1, wherein the metabolite is caprolactam.

3. The method of claim 1, wherein the metabolite is N-cyclohexylformamide.

4. The method of claim 1, wherein the metabolite is threo-3-hydroxy-2-methylbutyric acid.

5. The method of claim 1, wherein the metabolite is sulfoacetic acid.

6. The method of claim 1, wherein the metabolite is (2,7-dimethyloctahydro-1H cyclopenta[c]pyridin-4-yl)methanol.

7. The method of claim 1, wherein the metabolite is trans-3-indoleacrylic acid.

8. The method of claim 1, wherein the metabolite is pantothenic acid.

9. The method of claim 1, wherein the metabolite is diaveridine.

10. The method of claim 1, wherein the metabolite is (5-benzyl-3,6-dioxo-2-piperazinyl)acetic acid.

11. The method of claim 1, wherein the metabolite is dasytrichone.

12. The method of claim 1, wherein the metabolite is indole.

13. The method of claim 1, wherein the metabolite is leucoline.

14. The method of claim 1, wherein the metabolite is skatole.

15. The method of claim 1, wherein the metabolite is 6-hydroxycaproic acid.

16. The method of claim 1, wherein the metabolite is 1,2,6-hexanetriol.

17. The method of claim 1, wherein the metabolite is naphthalen-2-amine.

18. The method of claim 1, wherein the metabolite is 8-hydroxyquinoline.

19. The method of claim 1, wherein the metabolite is 2,2,6,6-tetramethyl-4-piperidinol.

20. The method of claim 1, wherein the metabolite is 1,5-naphthalenediamine.

21. The method of claim 1, wherein the metabolite is 1H-indole-2-carboxylic acid.

22. The method of claim 1, wherein the metabolite is (E)-dacarbazine.

23. The method of claim 1, wherein the metabolite is dinitrosopentamethylenetetramine.

24. The method of claim 1, wherein the metabolite is 5-hydroxyindole-3-acetic acid.

25. The method of claim 1, wherein the metabolite is 3-amino-5,7-dimethyl-1-adamantanol.

26. The method of claim 1, wherein the metabolite is 11-aminoundecanoic acid.

27. The method of claim 1, wherein the metabolite is kynurenine.

28. The method of claim 1, wherein the metabolite is N-hydroxy-L-tryptophan.

29. The method of claim 1, wherein the metabolite is 2-(methylamino)-1H benzo[de]isoquinoline-1,3(2H)-dione.

30. The method of claim 1, wherein the metabolite is 5-hydroxy-N-formylkynurenine.

31. The method of claim 1, wherein the metabolite is N-lauroylglycine.

32. A method comprising:
a) obtaining a sample of culture media in which one or more embryos has been cultured in vitro;
b) measuring from the sample an amount of at least four metabolites selected from the group consisting of: caprolactam, N-cyclohexylformamide, threo-3-hydroxy-2-methylbutyric acid, sulfoacetic acid, phenylalanine, (2,7-dimethyloctahydro-1H-cyclopenta[c]pyridin-4-yl)methanol, trans-3-indoleacrylic acid, DL-tryptophan, pantothenic acid, diaveridine, (5-benzyl-3,6-dioxo-2-piperazinyl)acetic acid, dasytrichone, indole, leucoline, skatole, 6-hydroxycaproic acid, 1,2,6-hexanetriol, naphthalen-2-amine, 8-hydroxyquinoline, 2,2,6,6-tetramethyl-4-piperidinol, 1,5-naphthalenediamine, 1H-indole-2-carboxylic acid, (E)-dacarbazine, dinitrosopentamethylenetetramine, 5-hydroxyindole-3-acetic acid, 3-amino-5,7-dimethyl-1-adamantanol, 11-aminoundecanoic acid, kynurenine, N-hydroxy-L-tryptophan, 2-(methylamino)-1H-benzo[de]isoquinoline-1,3(2H)-dione, 5-hydroxy-N-formylkynurenine, and N-lauroylglycine; and
c) comparing the amount of each measured metabolite in the sample to a reference of known clinical outcomes; and
d) transferring one or more embryos with a high probability of implantation success based on step c) into a subject's uterus.

33. A kit comprising an aliquot of each of 3 metabolites selected from the group consisting of: caprolactam, N-cyclohexylformamide, threo-3-hydroxy-2-methylbutyric acid, sulfoacetic acid, (2,7-dimethyloctahydro-1H-cyclopenta[c]pyridin-4-yl)methanol, trans-3-indoleacrylic acid, pantothenic acid, diaveridine, (5-benzyl-3,6-dioxo-2-piperazinyl)acetic acid, dasytrichone, indole, leucoline, skatole, 6-hydroxycaproic acid, 1,2,6-hexanetriol, naphthalen-2-amine, 8-hydroxyquinoline, 2,2,6,6-tetramethyl-4-piperidinol, 1,5-naphthalenediamine, 1H-indole-2-carboxylic acid, (E)-dacarbazine, dinitrosopentamethylenetetramine, 5-hydroxyindole-3-acetic acid, 3-amino-5,7-dimethyl-1-adamantanol, 11-aminoundecanoic acid, kynurenine, N-hydroxy-L-tryptophan, 2-(methylamino)-1H-benzo[de]isoquinoline-1,3(2H)-dione, 5-hydroxy-N-formylkynurenine, DL-tryptophan, phenylalanine, and N-lauroylglycine.
